(19) United States
(12) Patent Application Publication
(10) Pub. No.: US 2026/0199240 A1
(43) Pub. Date: Jul. 16, 2026

(54) BIOMEDICAL APPLICATIONS OF AZOBENZENE-BASED LIPIDOIDS

(71) Applicant: Trustees of Tufts College, Medford, MA (US)

(72) Inventors: Qiaobing Xu, Medford, MA (US); Yu Zhao, Medford, MA (US)

(21) Appl. No.: 19/133,926

(22) PCT Filed: Dec. 7, 2023
(86) PCT No.: PCT/US2023/082918

§ 371 Date: May 29, 2025

Related U.S. Application Data
(60) Provisional application No. 63/430,797, filed on Dec. 7, 2022.

(51) Int. Cl. A61K 9/1272; C07C 245/08

(52) U.S. Cl. CPC A61K 9/1272; C07C 245/08

(57) ABSTRACT

Disclosed are azo-based lipidoid molecules, and their use to form biomimetic lipidoid artificial compartments for bidirectional regulation of enzyme activity.

### RELATED APPLICATION

This application claims the benefit of priority to U.S. Provisional Patent Application No. 63/430,797, filed Dec. 7, 2022.

### GOVERNMENT SUPPORT

This invention was made with government support under EB027170-01 awarded by the National Institutes of Health (NIH). The government has certain rights in the invention.

### BACKGROUND

Precise regulation of the enzyme activity is important.⁶⁻¹⁰ Many photo-responsive inhibitors and inhibitor-enzyme conjugates have been developed to achieve on-and-off control of enzymatic activity.¹¹ In general, these enzymatic “switches” contain a photoisomerizable unit, with an inhibitor on one side. The meticulously designed “switch” adopts a particular geometry, which makes one of its isomeric forms more favorable than the other for properly docking of the inhibitor into the catalytic site.¹²⁻¹⁴ However, this method requires new inhibitors to be synthesized for each new enzyme via a trial-and-error process. On-demand activity control can also be achieved by site-selectively crosslinking two suitable residues of an enzyme with a photoisomerizable linker.¹⁵ Following photo-triggered isomerization of this linker, the conformation of the active site changes, thereby resulting in reversible changes in catalytic activity. To optimize this “switch effect”, a large number of enzyme variants have to be screened to determine the suitable residues for crosslinking. This screening process makes this method laborious and time consuming. As a simpler approach, directly grafting photo-responsive phase transition polymers onto the enzyme surface can reversibly interfere with the recognition between an enzyme and its substrates.¹⁶ However, one consideration is that these non-inhibitor-based approaches usually require genetic mutation and/or covalent modification of the native enzymes, which could result in irreversible loss of catalytic activity. Therefore, in view of practicality, a generic and facile approach that requires neither inhibitor screening nor mutation and modification of the enzymes is preferred.

### SUMMARY

In one aspect, provided are compounds formula (I) or formula (II):

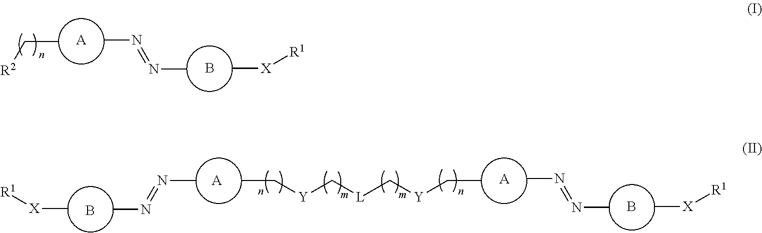

wherein,
A and B are each independently selected from aryl and heteroaryl;
X is independently selected from *—(C═O)NH—, *—NH(C═O)—, *—O(C═O)—, and *—(C═O)O—;
Y is independently selected from **—O(C═O)—, **—(C═O)O—, —N(R³)—, and —N⁺(R³)₂—;
L is absent or —NR⁴—;
R¹ is C₆₋₂₀ alkyl;
R² is independently selected from —OR⁵, —O(C═O)R⁵, —(C═O)OR⁵, —N(R³)₂ and —N⁺(R³)₃;
R³ is independently selected from H and C₁₋₆ alkyl;
R⁴ is independently selected from C₁₋₆ alkyl and C₁₋₆ alkylamino;
R⁵ is independently selected from C₁₋₆ alkyl and C₁₋₆ alkenyl;
n is independently 0-6;
m is independently 1-6;
* indicates the point of attachment to B; and
** indicates the point of attachment to (CH₂)ₘ.

### BRIEF DESCRIPTION OF THE DRAWINGS

FIG. 1 Lipidoid artificial compartments for bidirectional regulation of the enzyme activity through nanomechanical action. a) Synthesis route of Azo lipidoids. b) The structure of enzyme-encapsulated lipidoid artificial compartments. c) Schematic illustration of the permeability changes of the phospholipid membrane triggered by light irradiation for bidirectional regulation of the enzyme activity. The UV/Vis light stimulus induces continuous rotation-inversion movement of Azo lipidoids, resulting in an enhanced permeability of the membrane and the activation of encapsulated enzymes. Moreover, the membrane can self-revert to the impermeable state for enzyme activity inhibition as soon as light is removed.

FIG. 2 The reversible photoisomerization of Azo lipidoids. a) Schematic illustration of the trans-to-cis and cis-to-trans photoisomerization of Azo lipidoids. b) and c) The changes of UV-Vis absorption spectrum of Azo lipidoids in DMSO solution before and after irradiation with UV (365 nm for 6 s) and Vis light (>400 nm for 6 s), respectively. The power densities for 365 and >400 nm are 5.40 and 20 mW/cm², respectively. d) Diameter distributions of three different lipidoid artificial compartments suspended in HEPES buffer (50 mM, pH 7.2) obtained using DLS, together with their polydispersity indices (PDI). e) TEM images of different lipidoid artificial compartments. f), g) and h) UV-Vis absorption spectrum of different lipidoid artificial compartments including DMPC-Azo, DPPC-Azo, and DSPC-Azo in HEPES buffer after different treatments. The samples are irradiated with Vis, UV and UV/Vis light for 180 s at 37° C. i) Schematic illustration of the continuous rotation-inversion movement of Azo lipidoids in the presence of both UV (purple) and Vis (blue) light. j), k) and l) Thermal relaxation of different lipidoid artificial compartments in HEPES buffer at 37° C. The light was removed after irradiation for 30 min. The UV-Vis absorption at 340 nm was then recorded during the observed 96 h.

FIG. 3 Screening of the contents of Azo lipidoids for the preparation of carbonic anhydrase (CA)-encapsulated lipidoid artificial compartments. a), b) and c) Quantitative analysis of CA leakage from DMPC-Azo@CA, DPPC-Azo@CA and DSPC-Azo@CA with various percentages of Azo lipidoids after irradiation with UV/Vis light for 15 min in HEPES buffer at 37° C. d) Representative agarose gel electrophoresis result of the DSPC-Azo@CA with various percentages of Azo lipidoids after irradiation with UV/Vis light for 15 min. The CA is pre-labelled with fluorescent dye for visualization. Data are presented as mean±standard deviation (s.d.) from n independent experiments (n=3).

FIG. 4. Bidirectional regulation of the enzyme activity of CA by UV/Vis light. a) Schematic illustration of the competitive binding of PNPA with DNSA to the active site of CA during the UV/Vis irradiation. State I, impermeable state; State II, permeable state. DNSA, which fluoresces upon binding to the active site of CA, and is not fluorescent when kicked out from the active site. b) Fluorescence spectral changes (λₑₓ=280 nm) of CA (0.25 μM) and DNSA (5 μM)-encapsulated lipidoid artificial compartments in the presence of PNPA (250 μM) at 37° C. after different treatments. c) The fluorescence intensity of DNSA at 470 nm after different treatments. d) Kinetic parameters of free CA and different CA-encapsulated lipidoid artificial compartments after different treatments. e) Cycled off-on switching of the enzyme activity of DMPC-Azo@CA, DPPC-Azo@CA, and DSPC-Azo@CA in HEPES buffer at 37° C., respectively. CA-mediated enzymatic hydrolysis of PNPA to p-nitrophenol, exhibiting its characteristic absorption at 405 nm. The enzyme activities are estimated by measuring the reaction rate. f) The Tₘ and headgroup area of different helper lipids. g) The CA enzyme activity encapsulated in the compartments made with different helper lipids (various Tₘ and headgroup area) without UV/Vis light irradiation. h) The difference between the CA enzyme activity with or without UV/Vis light irradiation (the difference between the on and off state shown in FIG. 3e) in compartments with different helper lipids (various Tₘ and headgroup area). Data are presented as mean±standard deviation (s.d.) from n independent experiments (e, n=3; g and h, n=6).

FIG. 5. Bidirectional regulation of the catalytic activities of CAT and GOx. a) The O₂ generation in H₂O₂ (250 μM) solution after addition of DSPC-Azo@CAT (1 μg/mL, using CAT as standard), as measured by a portable dissolved oxygen meter. The sample solutions are exposed to the intermittent UV/Vis light (365 nm, 30 s; pink-shaded areas), alternately. b) The photograph showing the O₂ generation of DSPC-Azo@CAT (20 μg/mL, using CAT as standard) in H₂O₂ solutions (5 mM) before and after irradiation with UV/Vis light (90 s). c) The H₂O₂ concentration changes in glucose (1 mg/mL) solution after addition of DSPC-Azo@GOx (10 μg/mL, using GOx as standard). The sample solutions are exposed to the intermittent UV/Vis light (365 nm, 60 s; pink-shaded areas), alternately. d) Schematic illustration of the dialysis devices used to evaluate the capability of DSPC-Azo@GOx in controlled cell killing. e), f) and g) In vitro viabilities of 4T1 tumor cells received different treatments after 24 h. The cells are irradiated with UV/Vis light for 5 min every 8 hours. The power densities for 365 and >400 nm are 5.40 and 20 mW/cm², respectively. h) The cell morphology differences after different treatments (100 μg/mL, using GOx as standard). Data are presented as mean±standard deviation (s.d.) from n independent experiments (n=3).

FIG. 6. ¹H NMR spectrum of Compound 2 in CDCl₃, 500 MHz, 25° C.

FIG. 7. ¹H NMR spectrum of Compound 4 in CDCl₃, 500 MHz, 25° C.

FIG. 8. a) ¹H NMR spectrum of Azo lipidoid in CD₃OD, 500 MHz, 25° C. b) ¹³C NMR spectrum of Azo lipidoid in CD₃OD.

FIG. 9. a) The time required to reach the photostationary state in DMSO solution. b) The time required to reach the photostationary state in aqueous solution when the concentration of Azo-lipidoids is below the CMC (10 μM). c) The time required to reach the photostationary state in aqueous solution when the concentration of Azo-lipidoids is above the CMC (100 μM). d), e), and f) The time required to reach the photostationary state in HEPES buffer (50 mM, pH 7.2) at 37° C. g) and h) The changes of UV-Vis absorption spectrum of DSPC-Azo@CA in HEPES buffer before and after irradiation with UV (365 nm for 30 s) and Vis light (>400 nm for 30 s), respectively. The power densities for 365 nm and >400 nm are 5.40 and 20 mW/cm², respectively.

FIG. 10. a) The time required to reach the photostationary state under different Vis light intensity in HEPES buffer (50 mM, pH 7.2) at 37° C. b) UV-Vis absorption at 340 nm of different lipidoid artificial compartments including DMPC-Azo, DPPC-Azo, and DSPC-Azo in HEPES buffer. The samples are irradiated with UV/Vis light for 30 min at 37° C. Data are presented as mean±standard deviation (s.d.) from n independent experiments (n=3).

FIG. 11. Molecular dimensions of trans-Azo lipidoid and cis-Azo lipidoid.

FIG. 12. a) Transmission electron microscopy (TEM) images of three different lipidoid artificial compartments after UV/Vis light irradiation. b) Diameter distributions of different lipidoid artificial compartments before and after UV/Vis light irradiation. c) TEM image of DSPC-Azo@CA. d) The stability of lipidoid artificial compartments in HEPES buffer at 37° C. Data are presented as mean±standard deviation (s.d.) from n independent experiments (n=3).

FIG. 13. a) Illustration of separation of the free enzyme and compartment using centrifugal filtration. b) Quantitative measurements of CA adsorption on different lipidoid artificial compartments after incubation with CA solutions at 37° C. for 30 min. c) The encapsulation efficiency of lipidoid compartments with various helper lipid (DMPC, DPPC and DSPC). Data are presented as mean±standard deviation (s.d.) from n independent experiments (n=3).

FIG. 14. a) and b) Cycled off-on switching of the enzyme activity of DSPC-Azo@CAT and DSPC-Azo@GOx, respectively. c) Cell viability of 4T1 tumor cells received different treatments after 24 h. The cells are irradiated with UV/Vis light for 1, 2, 5, and 10 min every 8 hours, respectively. d) Cell viability of 4T1 tumor cells after 24 h incubation with various concentrations of Azo lipidoid. e) Cell viability of 4T1 tumor cells after 24 h incubation with various concentrations of DSPC-Azo (total lipid concentration was used in this case). In the 250 μg/ml DSPC-Azo solution, the Azo lipidoid concentration is 25 μM. Data are presented as mean±standard deviation (s.d.) from n independent experiments (n=3).

FIG. 15. Lipid-based nanomachine (LNM) opens endo-lysosomal compartments through nanomechanical action. a) Chemical structures of the two different Azo-based lipidoids, including SAzo lipidoid with a single tail and TAzo lipidoid with two tails. b) Schematic illustration of the LNM structure and its potential mechanism of overcoming intracellular barriers through light-triggered nanomechanical action. i) The LNM adheres onto the inner surface of endo-lysosomal membrane through electrostatic interaction after entering cells via endocytosis. ii) Reversible isomerization of the Azo unit induced by simultaneous UV and Vis light irradiation causes Azo-based lipidoids to undergo continuous rotation-inversion and stretch-shrink movements. Thus, LNMs containing Azo-based lipidoids destabilize the endo-lysosomal membrane and induce membrane disruption. iii) LNM opens the endo-lysosomal compartments and transports the cargoes to the cytoplasm.

FIG. 16. Photoisomerization of SAzo and TAzo lipidoids. a) Schematic illustration of the trans-to-cis and cis-to-trans photoisomerization of an Azo unit when irradiated with different lights. b) UV-Vis absorption spectrum of SAzo lipidoids before and after irradiation with UV (365 nm for 6 s) and Vis light (>400 nm for 6 s), respectively. c) UV-Vis absorption spectrum of TAzo lipidoids before and after irradiation with UV (365 nm for 6 s) and Vis light (>400 nm for 6 s), respectively. The power densities for 365 and >400 nm are 5.40 and 20 mW/cm², respectively. These studies were performed in DMSO solution. d) Diameter distributions of SAzo-LNM and TAzo-LNM in PBS (10 mM, pH 7.4) measured by DLS, together with the polydispersity index (PDI). e) TEM images of SAzo-LNM and TAzo-LNM. Scale bar, 200 nm. f) and g) UV-Vis absorption spectrum of SAzo-LNM and TAzo-LNM after different treatments. The samples are irradiated with Vis, UV and UV/Vis light for 180 s in PBS at 37° C., respectively. h) Schematic illustration of continuous rotation-inversion of SAzo or TAzo lipidoid caused by simultaneous UV (purple) and Vis (white) light irradiation.

FIG. 17. LNM transports mRNA from endo-lysosomal compartments to cytoplasm. a) Cell viability of HeLa cells after 24 h incubation with various concentrations of DOTAP-LNP, SAzo-LNM, TAzo-LNM, and Lpf2k, respectively. b) Fluorescence images of the cytosolic transport capabilities of DOTAP-LNP, SAzo-LNM, and TAzo-LNM in HeLa cells, respectively. Fluorescent labelled formulations are prepared (green) and incubated with cells. After 1 hour of incubation (allowing these formulations to enter cells), the cells were irradiated with UV/Vis light for 10 min. After another 30 min incubation, LysoTracker Red was added to stain the endo-lysosomal compartments. Scale bar, 10 μm. c) GFP mRNA transfection efficacy of DOTAP-LNP, SAzo-LNM, TAzo-LNM, Lpf2k, and naked GFP mRNA (1 μg mL-1, dotted line) tested on HeLa cells by quantifying GFP-positive cells. d) Molecular dimensions of SAzo lipidoid and TAzo lipidoid. Data are presented as mean±standard deviation (s.d.) from n independent experiments (n=3). Statistical significance was analyzed by one-way ANOVA with Tukey's multiple comparisons test. (***p<0.001, ****p<0.0001).

FIG. 18. LNM destabilizes the endo-lysosomal membrane and induces membrane disruption. a) Formulation details of LNMs with various percentages of SAzo and TAzo lipidoids (molar ratio, Azo units as standard). b) Fluorescence intensity of LysoSensor Green in HeLa cells after incubating with different LNMs and treating with or without UV/Vis light irradiation. c) and d) Detection of EEA-1, LAMP-2, and Cath-D in the cytoplasm of HeLa cells after different treatments using western blotting analysis. e) Schematic illustration of disruption of endo-lysosomal compartments and the release of EEA-1, LAMP-2, and Cath-D to the cytoplasm. f) Analysis of cytosolic transport efficiency of different formulations by SLEEQ assay. g) Schematic illustration of the experimental design. The mice were irradiated with UV/Vis light for 15 min at 1 h and 2 h after subcutaneous (SC) injection of mRNA-loaded LNMs. The power densities for 365 and >400 nm are 30 and 120 mW/cm2, respectively. h) Representative whole-body bioluminescence images of mice after SC injection of different formulations measured by the IVIS imaging system (0.25 mg kg-1 Luc mRNA, 5 μg per mouse). Images were taken at 5 h post-injection, and two mice in each group were shown. i) Relative luciferase expression in each group. Data are presented as mean±standard deviation (s.d.) from n independent experiments (b, n=4; f, n=5; i, n=3). Statistical significance was analyzed by one-way ANOVA with Tukey's multiple comparisons test. (*p<0.05, ***p<0.001, ****p<0.0001).

FIG. 19. LNM transports genome editing proteins from endo-lysosomal compartments to cytoplasm. a) Fluorescence images of HeLa cells after treatment with DOTAP-LNP/(−30)GFP-Cre, SAzo-LNM/(−30)GFP-Cre, and TAzo-LNM/(−30)GFP-Cre (1.5 μg mL-1 protein), respectively. Different formulations are incubated with cells. After 30 min incubation, the cells receive UV/Vis light irradiation for 10 min. After a further incubation, LysoTracker Red is added to stain the compartments. Scale bar, 10 μm. b) Quantitative analysis of co-localization of (−30)GFP-Cre with LysoTracker Red-labelled endo-lysosomal compartments (three independent experiments). The coefficients are close to 1 if they are highly colocalized. c) DsRed expression efficacy of DOTAP-LNP/(−30)GFP-Cre, SAzo-LNM/(−30)GFP-Cre, TAzo-LNM/(−30)GFP-Cre, and naked (−30)GFP-Cre (1.5 μg mL-1 protein) tested on HeLa-DsRed cells, respectively. d) Schematic illustration of delivery of Cre protein to delete the stop cassette and activate downstream DsRed protein. Data are presented as mean±standard deviation (s.d.) from n independent experiments (n=3). Statistical significance was analyzed by one-way ANOVA with Tukey's multiple comparisons test. (**p<0.01, ***p<0.001, ****p<0.0001).

FIG. 20. LNM transports tumour antigens from endo-lysosomal compartments to cytoplasm for DC-based immunotherapy. a) Schematic illustration of the experimental design. The bone marrow-derived dendritic cells (BMDCs) are incubated with LNM/OVA complexes for 6 hours, then received UV/Vis light irradiation for 10 min. After a further incubation, the BMDCs are collected. B16F10-OVA tumour-bearing C57/BL6 mice are employed and SC injected BMDCs (1×106 for per mouse). b) Representative flow cytometry analysis and c) Statistic analysis of the expression of H2kb-SIINFEKL in BMDCs after different treatments in vitro. d) Fluorescence images of the expression of H2kb-SIINFEKL complex (green) on BMDCs (red, staining with anti-CD11c antibodies). e) Flow cytometry analysis of the population of CD80+CD86+ BMDCs. f) Individual tumour growth curves after different treatments. g) Average tumour growth kinetics. h) Survival of B16F10-OVA tumour-bearing C57/BL6 mice. i) Flow cytometry analysis of the population of CD8+ T cells (gated on CD45+CD3+ cells) in tumour tissues after different treatments. j) Flow cytometry analysis of the expression of GzmBhigh in CD8+ T cells (gated on CD45+CD3+ cells) in tumour tissues. k) Flow cytometry analysis of the CD62LlowCD44high T cells (gated on CD3+CD8+ cells) in the spleens. l) Lungs collected from the mice after rechallenging with intravenous injection of B16F10-OVA tumour cells (1×106 cells per mouse). Data are presented as mean i s.d. from n independent experiments (c, d, i, j and k, n=3; f, g and h, n=6). Statistical significance was analyzed by one-way ANOVA with Tukey's multiple comparisons test for c, d, i, j, k, and two-way ANOVA with Tukey's test for g, log-rank (Mantel-Cox) test for h. (*p<0.05, **p<0.01, ***p<0.001, ****p<0.0001).

FIG. 21. Characterization of TAzo lipidoid. a) HPLC-ESI-MS spectrum of Compound 6. b) 1H NMR spectrum of TAzo lipidoid in CD₃OD, 500 MHz, 25° C. c) 13C NMR spectrum of TAzo lipidoid in CD₃OD.

FIG. 22. The photoisomerization of Azo-based lipidoids and their LNM formulations. a) and b) The time required to reach the photostationary state in DMSO solution. c) and d) The time required to reach the photostationary state under different Vis light intensity in DMSO solution. e) and f) The time required to reach the photostationary state in PBS at 37° C. Data are presented as mean±standard deviation (s.d.) from n independent experiments (n=3).

FIG. 23. Live-cell imaging experiments show the transport capability of LNMs (TAzo-LNMs). a) Fluorescence images of the cytosolic transport capabilities of NBD-labeled LNMs (TAzo-LNMs) when receiving UV/Vis light irradiation. b) Gray values of green channel (TAzo-LNMs) along with the corresponding yellow solid lines in the fluorescence images.

FIG. 24. Co-localization analysis of the transport capability of SAzo-LNMs and TAzo-LNMs. a) Flow cytometry analysis of the internalization of NBD-labeled formulations in HeLa cells. b) and c) Quantitative analysis of co-localization of NBD-labeled formulations with endo-lysosomal compartments labelled with LysoTracker Red. The coefficients are close to 1 if they are highly colocalized. Data are presented as mean±standard deviation (s.d.) from n independent experiments (n=3). Statistical significance was analyzed by one-way ANOVA with Tukey's multiple comparisons test. (***p<0.001, ****p<0.0001).

FIG. 25. Characterization of GFP mRNA-loaded SAzo-LNMs and TAzo-LNMs. a) Zeta potentials and b) particle size of different GFP mRNA-loaded formulations at five different N/P ratios (from 0.5 to 10). c) Agarose gel electrophoresis of GFP mRNA (2% TAE gel electrophoresis, 750 ng mRNA loaded for assay). d) UV-Vis absorption spectrum of GFP mRNA-loaded TAzo-LNMs after different treatments. The samples are irradiated with UV and Vis light for 30 s in PBS at 37° C., respectively. Data are presented as mean±standard deviation (s.d.) from n independent experiments (n=3).

FIG. 26. LNMs transport GFP mRNA from endo-lysosomal compartments to cytoplasm. a) Cell viability of HeLa cells after 24 h incubation with of GFP mRNA-loaded DOTAP-LNP, SAzo-LNM, TAzo-LNM, and Lpf2k (mRNA, 1 μg mL-1; formulation, 50 μg mL-1), respectively. b) GFP mRNA transfection efficacy of DOTAP-LNP, SAzo-LNM, TAzo-LNM, Lpf2k, and naked mRNA tested on HeLa cells by quantifying GFP-positive cells. Data are presented as mean±standard deviation (s.d.) from n independent experiments (n=3).

FIG. 27. LNMs destabilize the endo-lysosomal membrane and induce membrane disruption. a) and b) Detection of EEA-1, LAMP-2, and Cath-D in the cytoplasm. c) TEM images of SAzo-LNMs and TAzo-LNMs after UV/Vis irradiation. Scale bar, 100 μm. d) Representative whole-body bioluminescence images of mice after SC injection of Luc mRNA (0.25 mg kg-1, 5 μg per mouse) measured by the IVIS imaging system. Images were taken at 5 h post-injection. e) Images of western blotting analysis. Data are presented as mean±standard deviation (s.d.) from n independent experiments (n=3). Statistical significance was analyzed by one-way ANOVA with Tukey's multiple comparisons test. (*p<0.05, ****p<0.0001).

FIG. 28. LNMs transport Cre proteins from endo-lysosomal compartments to cytoplasm. a) Flow cytometry analysis of the internalization of LNM/(−30)GFP-Cre complexes (1.5 μg mL-1 protein) in HeLa cells after 30 min incubation. b) DsRed expression efficacy of DOTAP-LNP/(−30)GFP-Cre, SAzo-LNM/(−30)GFP-Cre, TAzo-LNM/(−30)GFP-Cre, and naked (−30)GFP-Cre (1.5 μg mL-1 protein) tested on HeLa-DsRed cells after 24 h incubation, respectively. Data are presented as mean±standard deviation (s.d.) from n independent experiments (n=3).

FIG. 29. Analysis of DC maturation. Representative flow cytometry analysis of the population of CD80+CD86+ BMDCs after different treatments.

FIG. 30. Antitumour effect in a mouse model of melanoma. a) hematoxylin and eosin (H&E) staining and cell proliferation antigen Ki-67 staining. b) The photographs of B16F10-OVA model tumours.

FIG. 31. Gating strategy for identification of lymphocytes. a) Gating strategy for identification of CD3+ T cells within the tumour tissues. b) Gating strategy for identification of CD8+ T cells in the spleens.

FIG. 32. Antitumour immunity in vivo. a) Representative flow cytometry analysis of the population of CD8+ T cells (gated on CD45+CD3+ cells) in tumour tissues after different treatments. b) Representative flow cytometry analysis of the expression of GzmBhigh in CD8+ T cells (gated on CD45+CD3+ cells) in tumour tissues. c) Representative flow cytometry analysis of the CD62LlowCD44high and CD62LhighCD44high T cells (gated on CD3+CD8+ cells) in the spleens. d) Flow cytometry analysis of the CD62LhighCD44high T cells (gated on CD3+CD8+ cells) in the spleens. Data are presented as mean±standard deviation (s.d.) from n independent experiments (n=3). Statistical significance was analyzed by one-way ANOVA with Tukey's multiple comparisons test. (**p<0.01, ***p<0.001, ****p<0.0001).

FIG. 33. Schematic diagram of the SLEEQ assay.

### DETAILED DESCRIPTION

In nature, biological systems evolve compartmentalization strategies for flexibly in regulating and maintaining metabolic reactions.¹⁷⁻¹⁸ Channel proteins on the surface of compartments allow cargoes, including small molecules and ions, to transport across compartment membranes.¹⁹ Most channel proteins regulate the transport of cargoes through undergoing a mechanical change (acting as “gates”).¹⁹⁻²⁰ For example, bacteria can reversibly open and close these “gates” to allow specific molecules to enter its interior, thereby mediating the flux of metabolites.²¹⁻²² Similar metabolic processes have also been found in eukaryotic cells and organelles.²³⁻²⁶ Inspired by these natural systems, encapsulation of the enzymes into an artificial compartment with “gates” on its membrane would be an innovative strategy to achieve the controllable enzyme activities. Importantly, neither inhibitor screening nor mutation and modification of the enzymes is necessary with this strategy.

After entering cells, most biologics are entrapped in endo-lysosomal compartments, resulting in the inability to enter the cytoplasm to function. Cationic lipid nanoparticles (LNPs) allow the cytosolic transport of these biologics by destabilizing the endo-lysosomal membranes, but have a low efficiency (<2%) due to the limited compartment disruption capability^{a1,a2}. The efficiency can be improved by increasing the (positive) surface charge density of cationic LNPs2. However, this change usually induces the destabilization of plasma membranes during internalization, resulting in severe cytotoxicity. Inspired by biological systems, we reported the use of the nanomechanical action from azobenzene (Azo) derivatives to mimic the “off-on” switching of channel proteins on the plasma membrane, which allowed for controlled transmembrane transport of small molecules^{a3}. Therefore, abandoning the traditional viewpoints of drug delivery and finding answers from nature may be a better approach to overcome the above-mentioned issues^{a4,a5}. Molecular machines are assemblies of a certain number of molecular components (usually proteins), which perform specific mechanical movements (outputs) through changing their conformation in response to appropriate external stimuli (inputs)^{a6,a7}. These molecular machines enable complex and delicate processes by executing rotational and translational movements at the cellular level, which are essential for regulating intracellular, transmembrane, and intercellular transport of substances^{a8-a10}. Kinesin, dynein, and their relatives are amazing examples of molecular machines. They covert chemical energy from adenosine-5′-triphosphate (ATP) hydrolysis into mechanical work along cellular microtubules for macromolecule transport¹¹. Unfortunately, these natural molecular machines can hardly handle the transport of exogenous biologics within cells, not to mention the transmembrane transport from endo-lysosomal compartments to the cytoplasm.

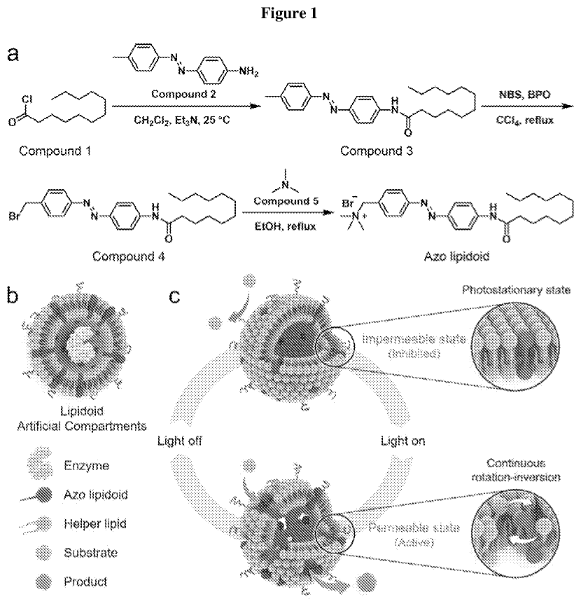

Herein a lipidoid artificial compartment and its capability to mediate bidirectional regulation of the enzyme activity using light is disclosed. A photoisomerable amphiphilic azobenzene (Azo) lipidoid was synthesized, and co-assembled with helper lipids to construct the artificial compartments (Scheme 1a-b). Azo lipidoids undergo a reversible isomerization between the extended trans- and compact cis-isomer when they are irradiated with ultraviolet (UV, 365 nm) and visible light (Vis, >400 nm).¹⁻⁴ This reversible isomerization of Azo lipidoids triggered by simultaneous ultraviolet (UV)- and visible (Vis)-irradiation causes the lipidoid to undergo a continuous rotation-inversion and stretch-shrink motions (FIG. 1a).⁵ Thus, Azo lipidoids can act as stirrers to enhance the permeability of the phospholipid membrane through nanomechanical action. Because of this, Azo lipidoids can function as “gates” that only open in the presence of UV and Vis (UV/Vis) lights. This architecture allows for controlled photo-regulation of membrane permeability, and spatiotemporally allows substrates to enter into its interior and products to be released.

Herein, a lipidoid-based artificial compartment composing photoisomerable Azo lipidoids and helper lipids was prepared.²⁷ As illustrated in Scheme 1c, the membrane of the artificial compartment is impermeable in the absence of UV/Vis light, which keeps the encapsulated enzymes in an inhibited state. However, the membrane becomes permeable upon UV/Vis light irradiation, thereby enabling the substrates to enter the compartment and carry out the enzymatic reaction. Moreover, this membrane can then self-revert to its impermeable state when light is removed, thereby stopping the reaction (re-inhibition of the enzymes). Herein, helper lipids with different phase transition temperatures (Tₘ) and head group areas, including dimyristoylphosphatidylcholine (DMPC), dipalmitoylphosphatidylcholine (DPPC), and distearoylphosphatidylcholine (DSPC), were employed. Among them, it was found that the artificial compartments prepared with DSPC lipids and Azo lipidoids showed great performance in reversibly regulating enzyme activity. The activity of carbonic anhydrase (CA) was increased by up to 9.5-fold after UV/Vis light irradiation when the enzyme was encapsulated in the compartment. Importantly, since neither genetic mutation nor chemical modification of the enzyme was required for enzymatic regulation, CA retained more than 80% of its activity after three cycles of activation and inhibition compared to its native counterpart. Furthermore, this biomimetic approach does not require inhibitors and could be a generic strategy for multiple enzymes. The effects of artificial compartments on off-on switch of the activities of catalase (CAT) and glucose oxidase (GOx) were studied.

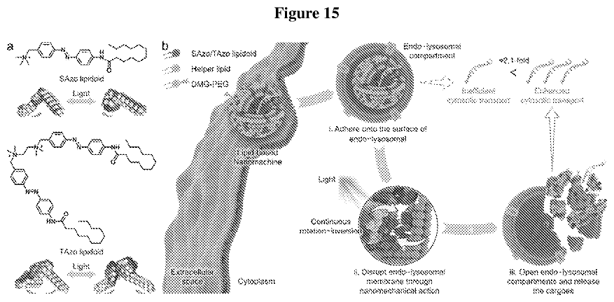

An artificial nanoscale molecular machine (nanomachine) was designed and fabricated by co-assembling photoisomerable amphiphilic Azo-based lipidoids and helper lipids, which achieves enhanced cytosolic transport of exogenous biologics by disrupting endo-lysosomal compartments through the nanomechanical action of azobenzene upon light irradiation. Unlike natural molecular machines, this lipid-based nanomachine (LNM) is designed to convert light energy into mechanical movements^{a12}. This is because photons are one of the most convenient energy inputs to drive LNMs. Specifically, Azo-based lipidoids in LNMs undergo the reversible isomerization between compact cis- and extended trans-isomers upon irradiation with ultraviolet (UV, 365 nm) and visible light (Vis, >400 nm, FIG. 15a). The isomerization of Azo-based lipidoids caused by simultaneous UV and Vis (UV/Vis) irradiation enables them to produce continuous rotation-inversion and stretch-shrink movements. Thus, after entering cells via endocytosis, LNMs interact with the endo-lysosomal membrane, and Azo-based lipidoids act as rotors upon irradiation with UV/Vis light, thereby enhancing destabilization and disruption of the membrane through nanomechanical action (FIG. 15b). In this work, a lipid formulation is chosen due to its capability in loading various cargoes. This architecture allows for efficient transport of exogenous biologics from endo-lysosomal compartments to the cytoplasm driven by consumption of photons (inputs). Our results showed that LNMs could enhance endo-lysosomal transport (escape) and facilitate efficient delivery of various biologics into the cytoplasm. It was found that both nucleic acid and protein cargoes could be transported to the cytoplasm using this LNM-based biomimetic approach, which provides a solution to improve the intracellular delivery of biologics for therapeutic applications.

To demonstrate the generality of LNMs and broaden their potential applications, its capability to carry tumour antigen from endo-lysosomal compartments to the cytoplasm and present the antigen on major histocompatibility complex class I (MHC-I, a critical recognition signal for stimulation of the cytotoxic T cells) in dendritic cells (DCs) was investigated. This is essential for achieving efficient antigen cross-presentation to enhance DC vaccine-based immunotherapy. It was found that LNMs could facilitate efficient tumour antigen cross-presentation and DC maturation when coupled with UV/Vis light irradiation. The results from animal experiments showed that administration of irradiated DCs pre-treated with LNM/tumour antigen complexes triggered enhanced cross-priming of cytotoxic T cells, thereby achieving robust antitumour immunity in a melanoma mouse model

A photoisomerable amphiphilic Azo lipidoid was synthesized, which consists of an Azo-based quaternary ammonium salt as the hydrophilic headgroup and a flexible alkyl chain as the hydrophobic tail. The photoisomerization kinetics of Azo lipidoid (50 μM) was monitored in dimethyl sulfoxide (DMSO) solution using UV-Vis absorption spectroscopy. As shown in FIG. 1b, upon UV light irradiation, the absorption of Azo lipidoid at 360 nm gradually decreased (corresponding to π-π* transition of the trans-isomer), while an increased absorbance at 440 nm was observed (corresponding to n-π* transition of the cis-isomer).³ These spectral changes leveled off after 6 s of irradiation with UV light because the photostationary state was reached (5.40 mW/cm²). Irradiation of Azo lipidoid with Vis light for additional 6 s induced an opposite spectral change (FIG. 1c), indicating that the Azo units could be easily isomerized, even in the cationic lipid form.

The critical micelle concentration (CMC) of Azo lipidoid was evaluated in aqueous solution. The Azo lipidoid has a relatively low CMC (≈30 μM). Moreover, isomerization of Azo lipidoids could occur either above or below the CMC when irradiated with the UV/Vis light. It should be noted that, the light intensity is a crucial parameter in this cis-trans isomerization process. Similar isomerization rates of Azo lipidoids are essential for creating and maintaining the continuous rotation-inversion movement. If the rates are not in equilibrium, the Azo lipidoids would tend toward a photostationary state.²⁸ Thus, a higher intensity of Vis light (20 mW/cm², 3.7 times as much as UV light) was chosen. The different intensities between two lights were due to the fact that Vis-induced cis-to-trans isomerization is harder than UV-induced trans-to-cis isomerization.²⁸⁻³²

Next, DMPC, DPPC, and DSPC with different Tₘ and headgroup areas were chosen as helper lipids. Three different artificial compartments were prepared by co-assembling Azo lipidoids with a helper lipid (DMPC, DPPC or DSPC), cholesterol, and 1,2-Distearoyl-sn-glycero-3-phosphoethanolamine-N-methoxy-poly(ethylene glycol) (mPEG-DSPE) (denoted as DMPC-Azo, DPPC-Azo, and DSPC-Azo, respectively). Cholesterol is used to improve the stability of the compartments. A PEG corona outside of compartments prevents surface adhesion of the enzymes. Dynamic light scattering (DLS) measurements showed that all artificial compartments had a diameter of around 150 nm, regardless of the helper lipid used (FIG. 1d). Transmission electron microscopy (TEM) images of these artificial compartments showed a closed vesicular morphology with bilayer structure (FIG. 1e). Moreover, the size of all three compartments over 24 h incubation in 37° C. by DLS was measured, and observed negligible size change, indicating the relatively high stability of these artificial compartments.

Then, the photoisomerization properties of the artificial compartments were monitored in HEPES buffer (50 mM, pH 7.2) at 37° C. The cis-rich and trans-rich photostationary states were reached after irradiation of artificial compartments with UV and Vis light for about 30 s, respectively. To investigate the continuous molecular rotation-inversion, the artificial compartments were irradiated with UV, Vis, and UV/Vis light for a relatively long time (180 s, 6-fold the time required to establish photostationary state), respectively. All samples showed similar variations in their absorption spectrum (Figure if-h). As expected, long-time UV or Vis irradiation enabled the Azo lipidoids to reach a cis-rich (low absorption at 340 nm, colored dotted lines) or trans-rich (high absorption at 340 nm, gray solid lines) photostationary states, respectively. When the UV/Vis light was used to irradiate samples, a moderate-intensity absorption (colored solid lines) was observed. Moreover, the absorption did not change as the time of irradiation prolonged (30 min). These results indicate that simultaneous UV- and Vis-irradiation enables the isomerization of Azo lipidoids to reach and maintain a metastable equilibrium state containing both cis- and trans-isomers (almost equivalent amount), which is out of the thermodynamic equilibrium.³³⁻³⁴. In this dynamic equilibrium state, a certain number of trans-isomers are converted into cis-isomers due to the presence of UV light, meanwhile, a similar number of cis-isomers are converted into trans-ones due to the presence of Vis light. Thus, the Azo lipidoids should undergo a continuous rotation-inversion movement (FIG. 1i). During this process, Azo lipidoids act as molecular stirrers accompanied with stretch-shrink motions, which have the potential to regulate the permeability of compartment membrane. In addition, these movements caused negligible changes in diameter size and morphology, as was observed after UV/Vis light irradiation, indicating the excellent structural stability of the compartments.

It was found that the time required to reach or restore the thermodynamic equilibrium state through thermal relaxation was much slower (>48 h) after removing the light at 37° C. (FIG. 1j-1l). The extremely slow conformational changes of Azo lipidoids during this period were not sufficient to alter the permeability of the compartment membranes. This suggests that the membrane permeability is significantly reduced after the light is removed. These results provide the solid structural foundation for bidirectional regulation of enzyme activity using lipidoid artificial compartments through nanomechanical action.

As a proof of concept, CA was chosen as a model enzyme. In order to achieve efficient activity regulation, the nonspecific surface adhesion of CA during the preparation process should be avoided. This is because the unencapsulated CA adsorbed on the surface of compartments would remain their activity consistently, resulting in ineffective regulation. Thus, the artificial compartments are designed to be covered with a PEG corona to reduce enzyme adsorption. To test this approach, the prepared artificial compartments were incubated with CA for 30 min, the mixture solutions were then filtered by centrifugal filtration and washed with HEPES buffer to remove the adsorbed CA.³⁵ Artificial compartments without PEG were prepared for comparison (denoted as DMPC-Azo-w/o PEG, DPPC-Azo-w/o PEG, and DSPC-Azo-w/o PEG, respectively). The results showed that negligible adsorption of CA was observed on the surface of PEGylated artificial compartments, whereas significantly higher levels of CA were adsorbed on compartments without PEG.

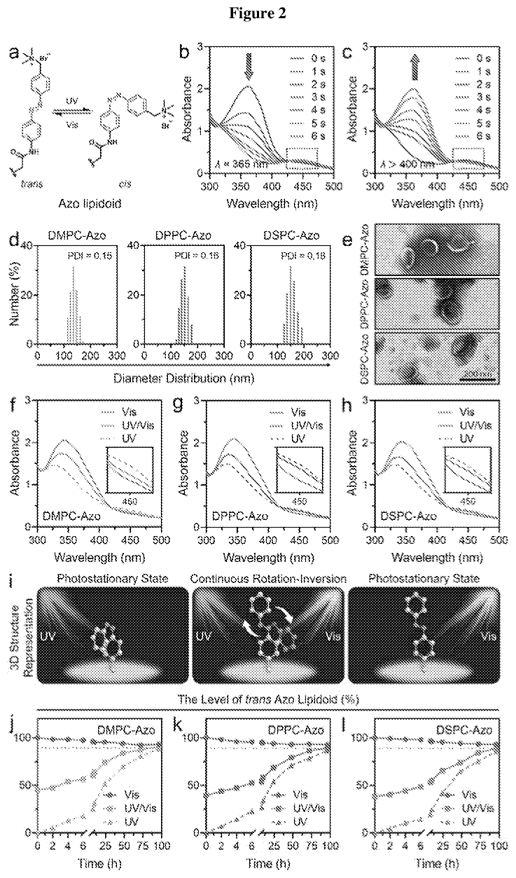

To achieve effective reversible regulation of enzyme activity, it is also essential to avoid CA leakage from the artificial compartments to the external solvent during UV/Vis light irradiation. It was hypothesized that the percentage of Azo lipidoid in the artificial compartments has a big impact on compartment stability. To test this hypothesis, a series of CA-encapsulated artificial compartments (DMPC-Azo@CA, DPPC-Azo@CA, and DSPC-Azo@CA, respectively; with encapsulation efficiency, ≈14%) with various percentages of Azo lipidoids (from 0% to 20%) were prepared. 1,2-dioleoyloxy-3-trimethylammonium propane chloride (DOTAP), which also contains a quaternary ammonium salt group (hydrophilic headgroup), was used as an alternative to Azo lipidoid for comparison. The same equivalent quaternary ammonium salt groups in each formulation could ensure the similar physical property of the compartments. It was found that encapsulation of the CA did not affect the nanostructure of artificial compartments, as well as the light-controlled trans-to-cis and cis-to-trans photoisomerization of Azo lipidoids. As shown in FIG. 2a-c, negligible CA leakage was observed when the percentage of Azo lipidoids was below 10% after irradiation with UV/Vis light for 15 min. When the Azo lipidoid content increased from 15% to 20%, a small amount of CA leaked from the interior of the artificial compartments. To visualize the CA leakage, agarose gel electrophoresis was performed to separate the leaked CA and the encapsulated CA. CA was pre-labeled with fluorescein isothiocyanate (FITC) and then encapsulated into compartments. In this study, DSPC-Azo@CA containing different percentages of Azo lipidoids were employed. As shown in FIG. 2d, nearly all of the CA is retained in the gel wells after light irradiation for 15 min (Azo lipidoids, <10%), confirming the negligible CA leakage. These results indicate that the biomacromolecules with nanometer size could not pass through the compartment membrane when the percentage of Azo lipidoids is lower than 10%. Thus, 10% Azo lipidoids in all formulations for subsequent studies was adopted.

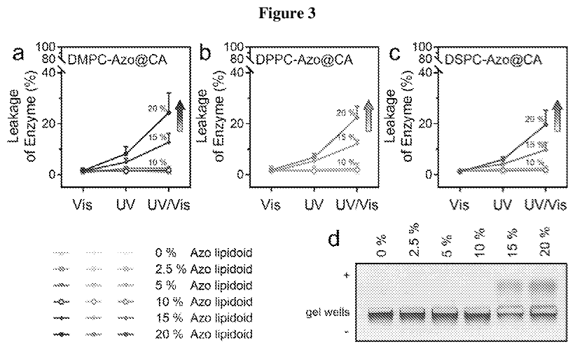

Then, it was investigated whether substrate molecules could enter the compartments to carry out enzymatic reactions. Dansylamide (DNSA, a dye molecule that can bind to the active site of CA) and p-nitrophenyl acetate (PNPA, a substrate of CA) were employed. DNSA fluoresced at 470 nm when bound to the active site (λex=280 nm). As illustrated in FIG. 3a, the CA-DNSA complexes were encapsulated into the artificial compartments, and the PNPA (250 μM) was then added into the external solvent. In theory, the compartment membranes would become permeable upon UV/Vis light irradiation, thereby allowing PNPA enter the compartments. Since the affinity of PNPA to CA is higher than DNSA, the DNSA pre-bound on the active site of CA should be replaced by PNPA resulting in a decrease in fluorescence intensity at 470 nm. When the CA-DNSA-encapsulated artificial compartments were exposed to the UV/Vis light for 180 s, a significantly reduced fluorescence intensity was observed (FIG. 3b, colored solid lines), suggesting that the PNPA entered the interior of compartments and bound to the active site of CA. In contrast, irradiation of these compartments by UV light only resulted in a moderate decrease of fluorescence intensity compared to UV/Vis light, which indicates that only a small amount of PNPA entered the compartments (colored dotted lines). This may be attributed to a transient change in membrane permeability caused by unidirectional conformational change of Azo lipidoids (trans-to-cis). These results demonstrate that the continuous rotation-inversion motion of Azo lipidoids plays an essential role in effectively changing the permeability of the compartment membranes, which allows substrates to enter the interior of the compartments.

Theoretically, the Vis irradiation should not induce a change in fluorescence intensity. This is because that most of the Azo lipidoids are in the trans-state under normal conditions, so irradiation with Vis light does not trigger the conformational changes of Azo units. However, it was found that the fluorescence intensity of DNSA decreased when DMPC-Azo@CA and DPPC-Azo@CA were irradiated with Vis light for 180 s, suggesting that even if the Azo lipidoid does not undergo a significant isomerization, PNPA still enters the DMPC-Azo@CA and DPPC-Azo@CA compartments (black dotted lines in FIG. 3b and FIG. 3c). These results are further confirmed by measuring the apparent Michaelis constant (K_{M}). As shown in FIG. 3d, encapsulation of CA into these artificial compartments resulted in the enhanced apparent K_{M} values compared to free CA. Significant decreases in K_{M} values were observed when the samples were exposed to UV/Vis light, suggesting that the increased accessibility of PNPA to CA is due to the enhanced permeability of the compartment membrane. As expected, the K_{M} values of DMPC-Azo@CA and DPPC-Azo@CA were much lower than DSPC-Azo@CA when they received Vis light treatment. This result confirms that some PNPA still reaches the active site of CA in the absence of UV/Vis light in the DMPC-Azo@CA and DPPC-Azo@CA groups. Therefore, DSPC is a better choice over the DMPC and DPPC for the preparation of artificial compartments to regulate enzyme activity.

The capability of the artificial compartments to reversibly regulate CA activity was investigated. The enzymatic hydrolysis of PNPA was monitored. To test this, CA-encapsulated artificial compartments without Azo lipidoids were prepared for comparison (denoted as DMPC@CA, DPPC@CA, and DSPC@CA, respectively). As shown in FIG. 3e, the repeated activation and (partial) inhibition of the enzyme activities were observed by irradiation with intermittent UV/Vis light in the DMPC-Azo@CA, DPPC-Azo@CA, and DSPC-Azo@CA groups. As a result, DSPC-Azo@CA was the most effective for reversibly regulating the activity of CA. The activity of DSPC-Azo@CA was inhibited (relative activity, 8.9%) in the absence of UV/Vis light, while increased by up to 86.0% (9.5-fold) after UV/Vis light irradiation (red solid line), indicating that the compartment membrane changed from an impermeable state to a permeable state under the UV/Vis light stimulus. In contrast, the enzyme activity of DSPC@CA without Azo lipidoid was inhibited throughout the experiments, and the intermittent irradiation did not affect the inhibition of activity suggesting the key role of Azo lipidoids in reversible regulation of CA activity. Although DMPC-Azo@CA and DPPC-Azo@CA showed the ability to reversibly regulate CA activity, they did not effectively inhibit the activity (relative activity, 34.9-42.0%). Non-complete inhibition of enzyme activity was also observed in the DMPC@CA and DPPC@CA groups, confirming that the compartments using helper lipid DMPC or DPPC are partially permeable.

The effect of the helper lipids on the compartment permeability could be explained by the different physical properties of the helper lipids, including Tₘ and headgroup surface area. The Tₘ of lipids determines the diffusion of molecules in bilayer membranes.³⁶⁻³⁷ Comparing with DSPC (Tₘ 55° C.), the lower Tₘ (DMPC, 24.1° C.; DPPC, 41.3° C.) allows the lipids to be in a fluidic liquid crystal phase, which allows for enhanced membrane permeability at 37° C.³⁸⁻⁴⁰ Moreover, the packing of the lipid chains becomes loose when the headgroup surface area of the lipid increases (DSPC, ~0.430 nm²; DMPC and DPPC, ~0.665 nm²),⁴¹ which could also result in enhanced permeability of the artificial compartment membrane (FIG. 3f). Thus, the DSPC with a higher Tₘ (55° C.) and smaller headgroup surface area provides improved compartment stability and prevents the passive diffusion of substrate into the compartments, as shown in FIG. 3g. As shown in FIG. 3h, comparing with DSPC-Azo@CA, both DMPC-Azo@CA and DPPC-Azo@CA showed smaller differences in enzyme activity with or without light irradiation (“on” and “off” state respectively). This can be attributed to the reduced permeability of the DSPC-Azo membrane to the substrate when there is no light irradiation, compared with DMPC-Azo and DPPC-Azo. These results show that both Azo lipidoids and helper lipids play crucial roles in controlling the permeability of the compartment membrane for enzyme activity regulation. Additionally, since neither genetic mutation nor chemical modification of the enzyme was required in this approach, the CA retained greater than 80% activity after three cycles of activation and inhibition (FIG. 3e, red solid line).

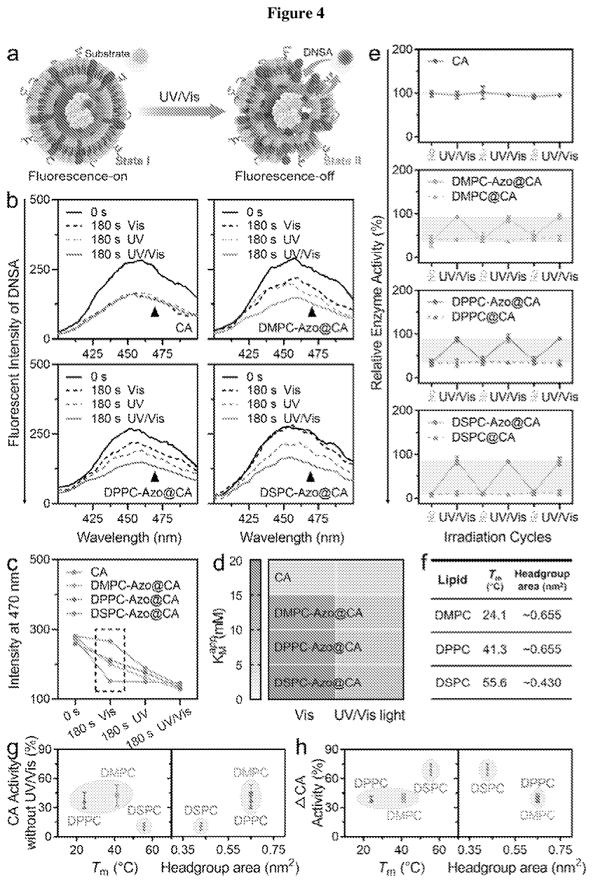

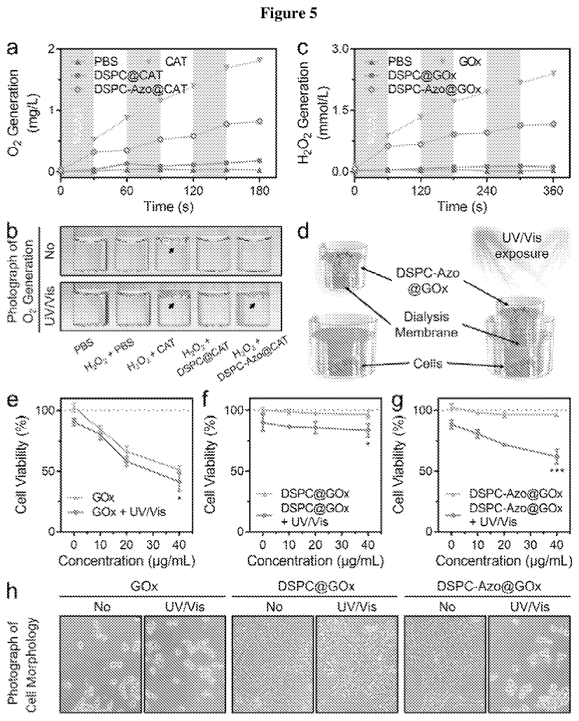

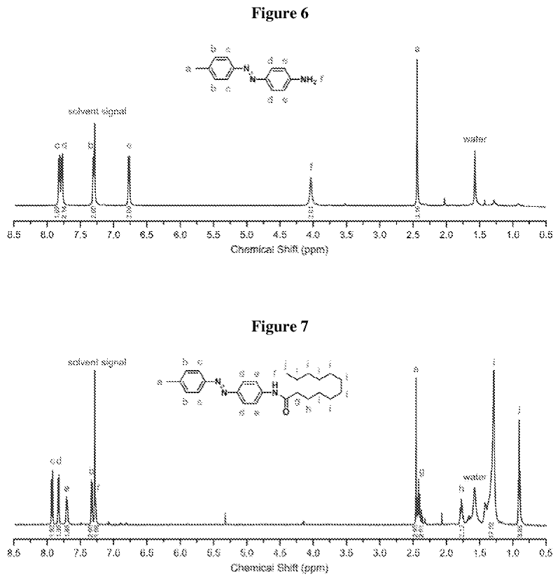

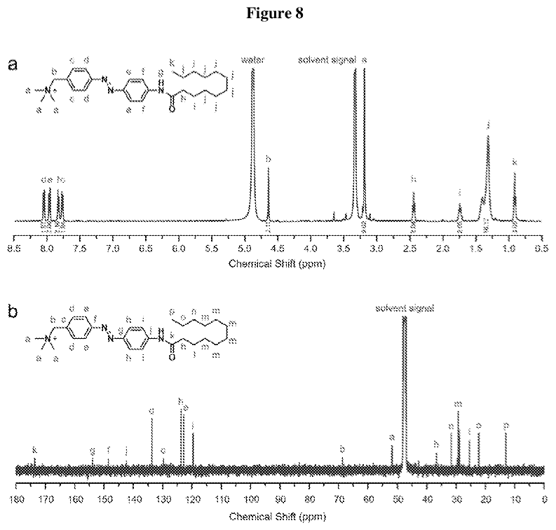

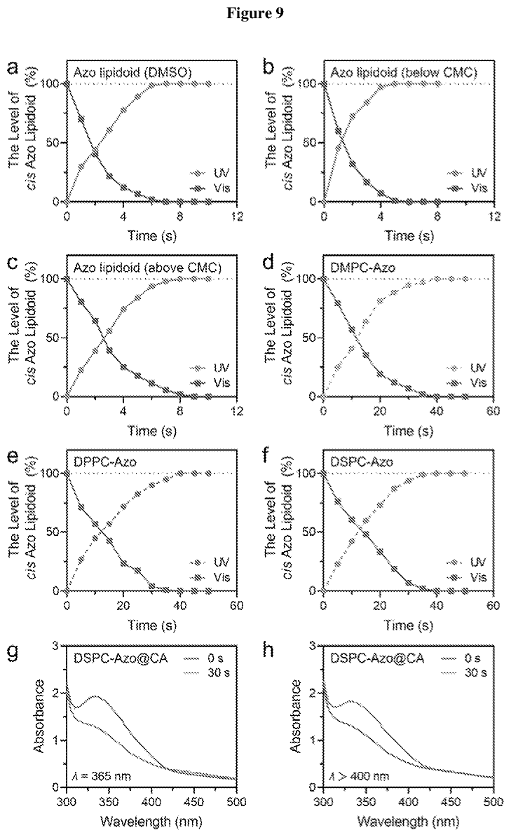

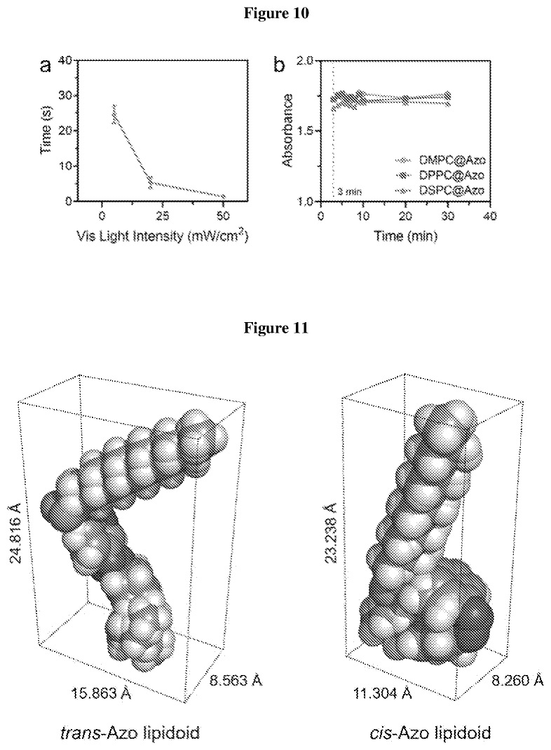

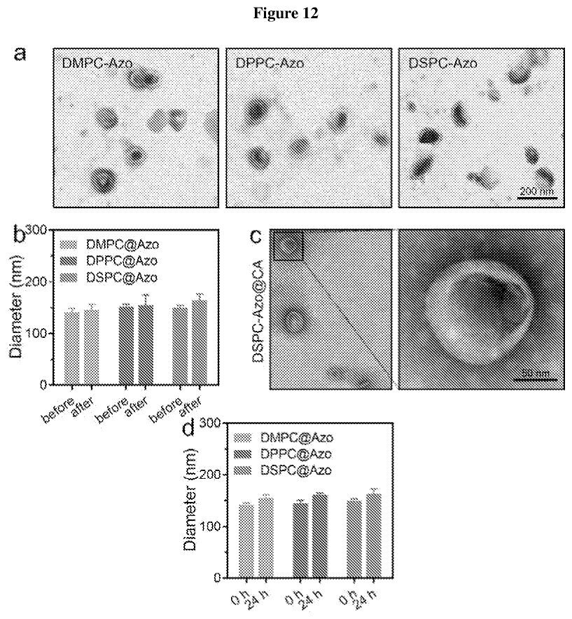

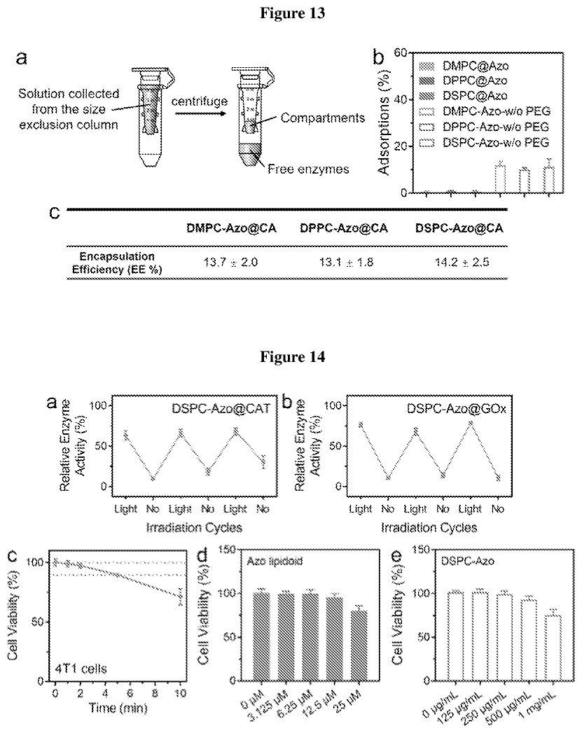

Given artificial compartments do not require inhibitors and can be an alternative control strategy for multiple enzymes, the capability of the DSPC-Azo compartments in bidirectional control of the activities of CAT and Gox was investigated. CAT is an enzyme that decomposes hydrogen peroxide (H₂O₂) into oxygen (O₂). In this study, CAT-encapsulated DSPC-Azo was prepared, and a formulation without Azo lipidoid was used for comparison (denoted as DSPC-Azo@CAT and DSPC@CAT, respectively). The generation of O₂ induced by UV/Vis light irradiation was monitored. As show in FIG. 4a, when the DSPC-Azo@CAT was added into H₂O₂ solution, the level of O₂ only increased in the presence of UV/Vis light (pink-shaded areas). In contrast, negligible O₂ generation was observed throughout the experiment in the DSPC@CAT group. This result was further confirmed by directly observing the generation of O₂ bubbles. As expected, there were lots of bubbles generated in the H₂O₂+DSPC-Azo@CAT group when the sample was exposed to the UV/Vis light for 5 min (FIG. 4b). These results indicated that the activity of DSPC-Azo@CAT could be switched on and off reversibly by UV/Vis light, confirming the “gate” role of the Azo lipidoid in this process.

GOx-based cancer starving therapy is known to inhibit tumor growth by depleting intratumoral glucose, which is oxidized into gluconic acid and H₂O₂.42 Moreover, the high concentrations of H₂O₂ can trigger tumor cell death.43-44 But GOx also consumes the glucose and generates H₂O₂ in healthy tissues due to the lack of tissue specificity. An ideal approach is that GOx would only be active at the desired site and period. Thus, GOx-encapsulated DSPC-Azo (denoted as DSPC-Azo@GOx) were prepared and its potential in controllably killing tumor cells was demonstrated. In this study, the generation of H₂O₂ induced by UV/Vis light irradiation was monitored. As a result, the H₂O₂ level only increased rapidly in the presence of UV/Vis light when DSPC-Azo@GOx was added into the glucose solution (FIG. 4c, pink-shaded areas, and S9b). In contrast, negligible H2O2 was detected throughout the experiment in the DSPC@GOx group (the formulation without Azo lipidoids).

Then, the on-demand activation of GOx in tumor blood vessels using a dialysis setup was simulated (FIG. 4d). The upper chamber was used as the simulated tumor blood vessels and the dialysis membrane (molecular weight cut off, 100 kDa) as vessel wall. 4T1 tumor cells were seeded in the basolateral chamber. DSPC-Azo@GOx was added into the upper chamber, followed by irradiating with UV/Vis light. The chambers were then irradiated for 5 min every 8 hours, because the prolonged UV irradiation results in cell damage and death.⁴⁵ As shown in FIG. 4g, DSPC-Azo@GOx showed remarkable cytotoxicity to cancer cells (GOx, 40 μg/mL) when exposed to the UV/Vis light, whereas negligible killing capacity was observed in the absence of UV/Vis light. There were no significant differences in cell viabilities in the GOx and DSPC@GOx group, regardless of whether they received the UV/Vis light treatment or not (FIG. 4e-f). Photographs of cell morphologies after different treatments confirmed this result. As shown in FIG. 4h, significant cell damage and death were observed after UV/Vis light irradiation in the presence of DSPC-Azo@GOx (GOx, 100 μg/mL). These results show that treatment of tumor cells with DSPC-Azo@GOx achieves photo-controlled cell killing.

In addition, the cytotoxicity study of both Azo lipidoid and DSPC-Azo was conducted. The dose dependent cell viability was measured in 4T1 cells, and the results showed the relatively good biocompatibility of these synthetic lipidoid and its formulations.

Inspired by biological membrane systems, a biomimetic lipidoid artificial compartment composed of photoisomerable Azo lipidoids and commercial helper lipids, which can bidirectionally regulate enzyme activity using light has been developed. The reversible photoisomerization of Azo lipidoids triggered by UV/Vis light creates a continuous rotation-inversion movement, accompanied with stretch-shrink motions. During this process, Azo lipidoids act as stirrers and enhance the membrane permeability through nanomechanical action. Moreover, the membrane reverts to its impermeable state when light is removed. Thus, this architecture achieves photo-regulation of the molecular flow passing through its membrane, thereby spatiotemporally allowing substrates to enter its interior. In this sense, Azo lipidoids have similar functions to channel proteins found in biological membrane systems that can regulate and maintain proper enzyme-mediated metabolic reactions. In this work, three different enzymes, including CA, CAT, and GOx, and their substrates (molecular weight, 34.0-181.2) are employed. By encapsulating these enzymes into artificial compartments, repeated activation and inhibition of the enzyme activities by intermittent UV/Vis light are achieved. Since neither mutation nor modification are required, negligible loss of the enzyme activity is observed. Moreover, this biomimetic strategy does not require inhibitors and is suitable for multiple enzymes, which may broaden the applications of enzymes in biological mechanism research and disease treatments. Additionally, our study also provides a viable route to fabricate cellular membrane mimics via simple chemistry, which has great values in mimicking cellular communication, intracellular (cascade) enzymatic reactions, as well as constructing the artificial protocells.⁴⁶

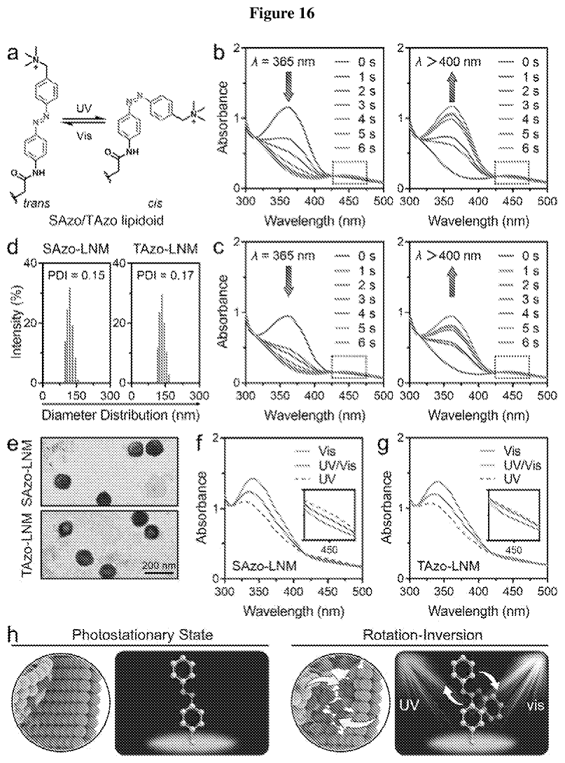

Two different photoisomerable amphiphilic Azo-based lipidoids, SAzo and TAzo lipidoids, were synthesized. As demonstrated in FIG. 15a, both lipidoids consist of Azo quaternary ammonium salts as the hydrophilic headgroup and flexible alkyl chains as the hydrophobic tail, where the SAzo lipidoid has a single tail and the TAzo lipidoid has two tails. The photoisomerization kinetics of SAzo lipidoids (30 μM) and TAzo lipidoids (15 μM) in dimethyl sulfoxide (DMSO) solutions using both ultraviolet and visible (UV-Vis) absorption spectrophotometry was investigated. As shown in FIGS. 16b and 16c, when SAzo and TAzo lipidoids were exposed to UV light (365 nm, 5.40 mW/cm²), the absorbance at 360 nm decreased gradually (corresponding to the π-π* transition in the trans-form) with the increase of UV irradiation time, while the absorbance at 440 nm increased (corresponding to the n-π* transition in the cis-form)^{a13}. Further experimentation showed that these spectral changes leveled off after 6 s of UV exposure due to the establishment of the photostationary state. Subsequently, an additional 6 s of Vis light (>400 nm) irradiation reversed these spectral changes, suggesting that the Azo units underwent rapid isomerization, even after modification with quaternary ammonium groups and alkyl chains. During the process of cis-trans isomerization, irradiation intensities were crucial parameters that directly determined the isomerization rates. In order to create and maintain a continuous rotation-inversion movement with these Azo units, similar trans-to-cis and cis-to-trans isomerization rates are necessary. This is because SAzo and TAzo lipidoids tend toward a photostationary state if the isomerization rates are not in equilibrium. According to the results, there was an optimal intensity of Vis light (20 mW/cm², 3.7 times that of UV intensity) for achieving similar isomerization rates. This difference between UV and Vis light intensities could be attributed to the fact that Vis-triggered cis-to-trans isomerization is more difficult than UV-triggered trans-to-cis isomerization14.

Next, two different LNMs were prepared by co-assembling SAzo or TAzo lipidoids with 1,2-dioleoyl-sn-glycero-3-phosphoethanolamine (DOPE), cholesterol (Chol), and 1,2-dimyristoyl-rac-glycero-3-methoxypolyethylene glycol-2000 (DMG-PEG) (molar ratio of 30:30:36.5:3.5, Azo units as standard, denoted as SAzo-LNM and TAzo-LNM, respectively)^{a15-a17}. Dynamic light scattering (DLS) measurements showed that SAzo-LNMs and TAzo-LNMs had similar diameters of approximately 130 nm in phosphate buffer (PBS, 10 mM, pH 7.4) (FIG. 16d). Both LNMs exhibited spherical morphologies as observed by transmission electron microscopy (TEM) (FIG. 16e). Next, their photoisomerization kinetics were measured in PBS at 37° C. According to the results, these two LNMs easily reached the cis- or trans-rich photostationary states after being irradiated with UV or Vis light for about 30 s, respectively. Compared to the time required (6 s) in DMSO solutions, the longer time might be attributed to the relatively crowded environment in an assembly formulation.

To investigate the continuous rotation-inversion movement, LNMs were exposed to UV, Vis, and UV/Vis light for 180 s, respectively, which was 6 times longer than the time required to reach the photostationary states. As shown in FIGS. 16f and 16g, long-time UV irradiation enabled SAzo and TAzo lipidoids to reach a cis-rich state (low absorbance at 340 nm, colored dotted lines), while Vis irradiation enabled them to reach a trans-rich state (high absorbance at 340 nm, gray solid lines). When these sample were exposed to UV/Vis light for 180 s, moderate-intensity absorptions (colored solid lines) were observed. These results suggested that co-irradiation with UV and Vis light led to simultaneous cis- and trans-isomerization of Azo-based lipidoids to reach and maintain a metastable equilibrium state containing nearly equal amounts of cis- and trans-forms, rather than a photostationary state. In this metastable equilibrium state, a certain amount of trans-isomers should be converted into the cis-forms because of UV irradiation, while a similar amount of cis-isomers should be converted into trans-forms from Vis irradiation. During this process, Azo-based lipidoids in LNMs should undergo continuous rotation-inversion and stretch-shrink movements in the presence of UV/Vis light, as demonstrated in FIG. 16h. As a result, Azo-based lipidoids show the potential to function as rotors to destabilize and disrupt the endo-lysosomal membrane through nanomechanical action, thereby enabling efficient transmembrane transport of biologics within cells (FIG. 15b).

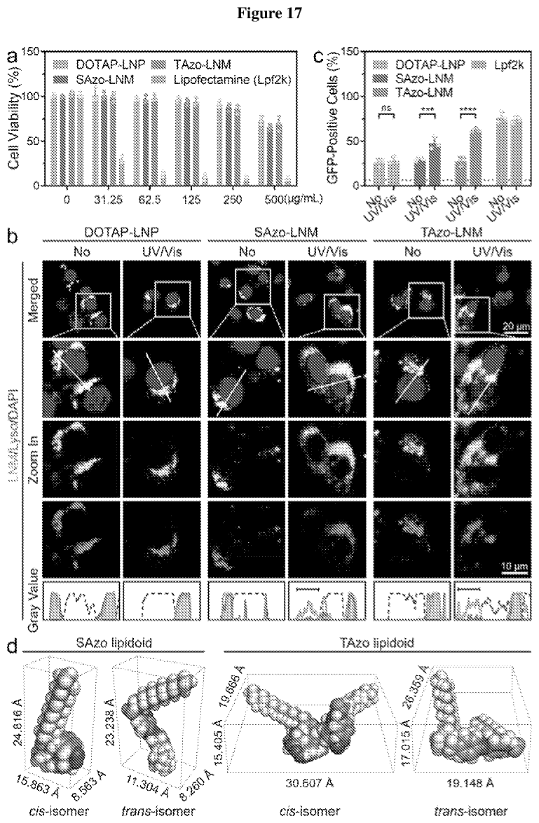

Various clinical applications can be achieved through delivery of mRNAs expressing antigens of cancers or infectious diseases, as well as disease-related therapeutic proteins^{a18, a19}. The recent success of two mRNA vaccines produced by Pfizer/BioNTech and Moderna for preventing coronavirus 2019 (COVID-19) further highlights the enormous potential of mRNA-based therapies to revolutionize life-science and medical research^{a20-a24}. Thus, as a proof-of-concept, green fluorescent protein (GFP) mRNA was chosen as a model cargo and the capability of LNMs to enhance transfection efficiency was studied. A formulation without Azo-based lipidoids was prepared as negative control, where 1,2-dioleoyloxy-3-trimethylammonium propane chloride (DOTAP) that also has a quaternary ammonium salt group was employed as an alternative to the Azo-based lipidoid (denoted as DOTAP-LNP). The biocompatibility of LNMs was first examined. The dose-dependent cell viability was measured in HeLa cells. As shown in FIG. 17a, negligible cytotoxicity of LNMs was observed at concentrations below 500 μg mL-1 after a 24 h incubation period, suggesting relatively good biocompatibility of these synthetic formulations. In contrast, the commercial transfection reagent Lipofectamine 2000 (Lpf2k) showed significant cytotoxicity under the same incubation conditions.

Next, the capability of LNMs to reach the cytoplasm from endo-lysosomal compartments was studied. Fluorescent-labeled SAzo-LNMs, TAzo-LNMs, and DOTAP-LNPs were prepared by using L-α-phosphatidylethanolamine-N-(4-nitro-benzo-2-oxa-1,3-diazole) (PE-NBD, a fluorescent lipid). These LNMs were then incubated with HeLa cells for 1 hour, which was sufficient for them to be taken up by cells. Then, the cells received UV/Vis light irradiation for 10 min. After another 30 min incubation, the cells were stained with LysoTracker Red and observed by confocal laser scanning microscopy (CLSM). SAzo-LNM and TAzo-LNM (green) could efficiently reach the cytoplasm from endo-lysosomal compartments (red) upon exposure to UV/Vis light. In the absence of UV/Vis light, these LNMs remained trapped in the endo-lysosomal compartments^{a25,a26}. Moreover, live-cell imaging experiments showed that the transmembrane efficiencies of these fluorescent-labeled LNMs were irradiation time-dependent^{a27}. With an increase of irradiation time, the signal of LNMs in the endo-lysosomal compartments decreased, while the signal of LNMs in cytoplasm increased. In contrast, almost all DOTAP-LNPs were entrapped in endo-lysosomal compartments regardless of the use of UV/VIS irradiation.

To further confirm the above results, co-localization analysis based on CLSM images was performed. Pearson's co-localization coefficient (PCC) between the fluorescence signals of different PE-NBD-labeled formulations and LysoTracker Red were calculated, respectively. PCC would be close to 1 if these two signals are highly co-localized. According to the results, the PCC values in the SAzo-LNM and TAzo-LNM groups are lower in the presence of UV/Vis irradiation (PCCSAzo-LNM=0.80; PCCTAzo-LNM=0.71), compared with these samples without irradiation (PCCSAzo-LNM=0.90; PCCTAzo-LNM=0.87. In contrast, the PCC value in the DOTAP-LNP group is close to 1 (PCCDOTAP-LNP=0.93) even when exposed to UV/Vis light. These results confirmed the light-enhanced endo-lysosomal escape effects and the crucial role of Azo-based lipidoids in the light-induced membrane disruption. It was found that the PCC value in the TAzo-LNM group is lower than that of the SAzo-LNM group, suggesting that TAzo-lipidiods may have a higher endosomal escape efficiency. This speculation is further corroborated in the following transfection efficiency and mechanism studies.

The capability of LNMs to facilitate GFP mRNA transfection by disrupting the endo-lysosomal compartments with light irradiation was investigated. The particle size and zeta potentials of GFP mRNA-loaded LNMs were first examined. Different formulations loaded with GFP mRNA at five different nitrogen/phosphorus (N/P) molar ratios (from 0.5 to 10) were prepared using the ethanol dilution method^{a28}. A slight increase in particle size was observed when loaded with GFP mRNA. In terms of zeta potentials, SAzo-LNM and TAzo-LNM followed the same profile in function of the N/P ratio. At low N/P ratio, such as 0.5 and 1.25, the formulations were negatively charged, while the charge proportionally increased with the increase of N/P ratio. Based on these results and previous studies, an N/P molar ratio of 2.5 was selected for further experiments29. Moreover, it was found that mRNA loading did not affect the light-controlled reversible photoisomerization of Azo-based lipidoids. Next, different mRNA-loaded formulations were incubated with cells for 4 hours, then irradiated with light for 10 min. Lpf2k and naked GFP mRNA were used as positive and negative controls, respectively. As shown in FIG. 17c, light irradiation resulted in a 1.6-fold and 2.3-fold increase in transfection efficiency in the SAzo-LNM and TAzo-LNM groups respectively. The transfection efficiency of the TAzo-LNM group is comparable to that of Lpf2k. However, relatively low transfection efficiencies and negligible differences were observed in DOTAP-LNP group with or without irradiation. These results suggest that light-irradiation improves the efficiency of LNMs containing Azo-based lipidoids for transporting GFP mRNA into the cytoplasm for GFP expression. More interestingly, it was found that the mRNA-loaded LNMs showed negligible cytotoxicity against HeLa cells (>90%, mRNA, 1 μg mL-1; formulation, 50 μg mL-1), while the mRNA/Lpf2k complex showed significant cytotoxicity (32.0%. These results showed good tolerability of these LNMs.

A study on the molecular dimensions of SAzo and TAzo lipidoids in their cis- and trans-conformations was conducted (FIG. 17d). The results showed that TAzo lipidoids undergo greater changes in their molecular height, length, and width compared to SAzo lipidoids during isomerization. Such dramatic changes in molecular dimensions may lead to increased disruption of endo-lysosomal membranes upon light-induced nanomechanical action of LNMs.

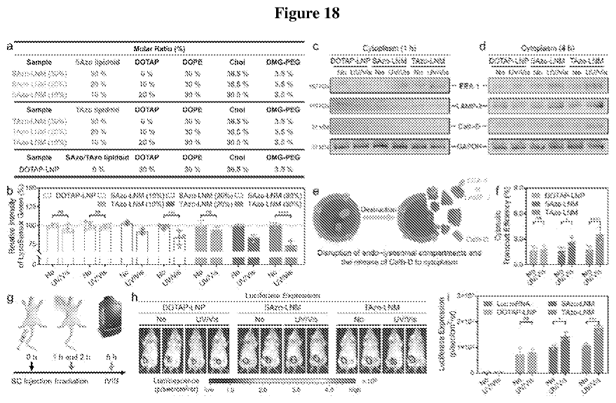

To further confirm this theory, a series of SAzo-LNMs and TAzo-LNMs with reduced percentages of Azo-based lipidoids (20% and 10%) were prepared, respectively (FIG. 18a). DOTAP was employed in these formulations as an alternative to Azo-based lipidoids to ensure that each formulation contained equivalent quaternary ammonium salt groups, and had similar physical properties. In this study, cells were incubated with different formulations, exposed to UV/Vis light, and stained by LysoSensor Green30. It is shown that destabilization or disruption of the endo-lysosomal compartments typically suppresses the LysoSensor signal^{a31,a32}. This is because LysoSensor probes rely on the decreased pH values to sense the endo-lysosomal compartments, and the protons that create a low pH environment is released into the cytoplasm upon the compartment rupture^{a33,a34}. Moreover, LysoSensor probes exhibit a pH-dependent increase in fluorescence intensity, and are almost non-fluorescent when outside acidic compartments^{a35}. Based on this, the integrity of endo-lysosomal compartments by measuring the fluorescence intensity of LysoSensor Green was studied. As shown in FIG. 18b, the fluorescence intensity in the TAzo-LNM (30%) group decreased to 73.4% after exposure to UV/Vis light compared to without light. And the fluorescence intensity decreased to 83.6% in the SAzo-LNM (30%) group under the same conditions. These results showed that a more distinct destabilization of endo-lysosomal compartments occurred in the TAzo-LNM (30%) group compared to that of the SAzo-LNM (30%) group, which is consistent with the mRNA transfection efficiency results (FIG. 17c). Moreover, when Azo-based lipidoid percentages in LNMs decreased from 30% to 10%, the compartment destabilization was gradually reduced, further confirming the critical role of Azo-based lipidoids in LNM-mediated transmembrane process.

To further investigate the effects of light irradiation on endosomal and lysosomal compartment disruption, western blotting immunoassay to evaluate whether specific protein markers were released from the endosomal (early endosomal antigen-1 (EEA-1)) and lysosomal (lysosome-associated membrane protein 2 (LAMP-2)) compartments to the cytoplasm was investigated^{a36,a37}. The cells were incubated with different LNMs for 1 hour or 4 hours, respectively, and then exposed to UV/Vis light. It was observed that the release of both EEA-1 (FIG. 17c) and LAMP-2 (FIG. 4d) were enhanced in the LNM groups irradiated with light. In contrast, there was no significant difference in protein release between DOTAP-LNM groups that did or did not receive light irradiation. Our results also showed that incubation of cells with SAzo-LNMs and TAzo-LNMs for 1 hour coupled with UV/Vis irradiation only resulted in the release of EEA-1 into the cytoplasm, and incubation of cells for 4 hours significantly enhanced the release of LAMP-2. It is known that nanoparticles enter endosomal compartments upon being taken up by cells in the first 1 to 2 hours, and are then transferred into the lysosomal compartments after 4 hours1. This explains why it was observed that the enhanced EEA-1 release after 1 hour of incubation, followed by enhanced LAMP-2 release after 4 hours of incubation. The release of Cathepsin D (Cath-D), a lysosomal protease, was also evaluated to confirm the enhanced lysosomal disruption upon light irradiation. Cath-D release in the first hour regardless of light irradiation (FIG. 17c) was not observed, while increased Cath-D release was observed after 4 hours of incubation (FIG. 17d), confirming the LNM-mediated lysosomal compartment disruption. These results suggested that the nanomechanical action of Azo-based lipidoids could trigger the effective disruption of endosomal and lysosomal compartments. TEM images also showed that UV/Vis irradiation loosened the nanostructure of LNMs possibly due to the stretch-shrink movements of Azo-based lipidoids.

Next, to quantify the cytosolic transport efficiency of LNMs, a recently reported quantification technique called the Split Luciferase Endosomal Escape Quantification (SLEEQ) assay was employed^{a38}. The SLEEQ assay is based on a highly sensitive bioluminescent split luciferase system that is composed of two subunits: a large BiT protein (LgBiT, 17.8 kDa) and a high affinity complementary peptide (HiBiT, 1.3 kDa). When separated these fragments have no luminescent activity, but when brought together, they form a functional enzyme that binds to a substrate which produces bright luminescence. HeLa cells were transfected with a LgBiT expression vector. LgBiT expressing cells were then incubated with HiBiT-loaded LNMs for 4 hours and received 10 min of UV/Vis light irradiation. The release of HiBiT into the cytoplasm generated a luminescencent signal, where the signal intensity correlated with the efficiency of endo-lysosomal release. According to the results, the cytosolic transport efficiency of the SAzo-LNM group increased 1.6-fold from 1.6% to 2.6%, and the TAzo-LNM group transport efficiency increased 2.1-fold from 1.7% to 3.6% upon exposure to irradiation (FIG. 18f).

The effects of light irradiation of the LNMs on the mRNA delivery efficiency in vivo were investigated. Firefly luciferase mRNA (Luc mRNA) was encapsulated in different LNMs, which the expression of luciferase could be visualized in vivo using the IVIS imaging system (PerkinElmer). Luc mRNA in a DOTAP formulation was used as control for comparison. The injection site was irradiated with UV/Vis twice (1 hour and 2 hours after receiving the injection) (FIG. 18g). As shown in FIG. 18h, the UV/Vis light irradiation significantly improved the bioluminescence signal in both the SAzo-LNM and TAzo-LNM groups, compared with the groups without light irradiation. In contrast, negligible differences of luminesence signal was observed in the DOTAP formulation regardless of light irradiation. Further quantitative analysis showed that UV/Vis irradiation resulted in 1.3-fold and 1.6-fold increase in transfection efficiency in the SAzo-LNM and TAzo-LNM groups, respectively (FIG. 18i). These results suggested that mRNA could be transported to cytoplasm with a higher efficiency using an LNM-based biomimetic approach both in vitro and in vivo.

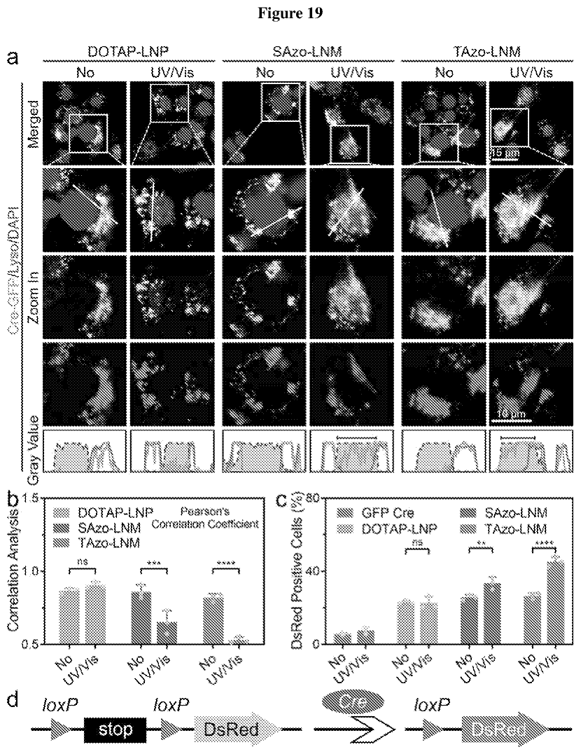

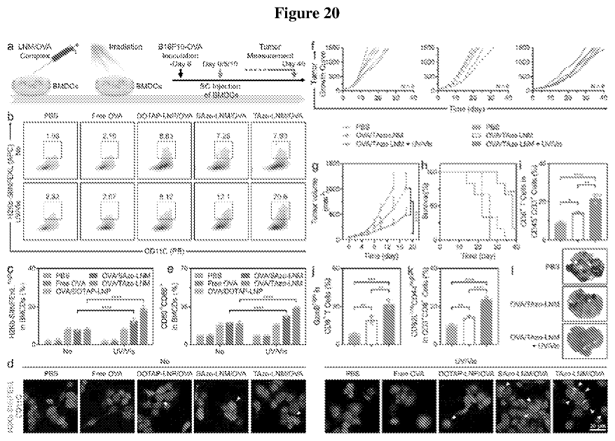

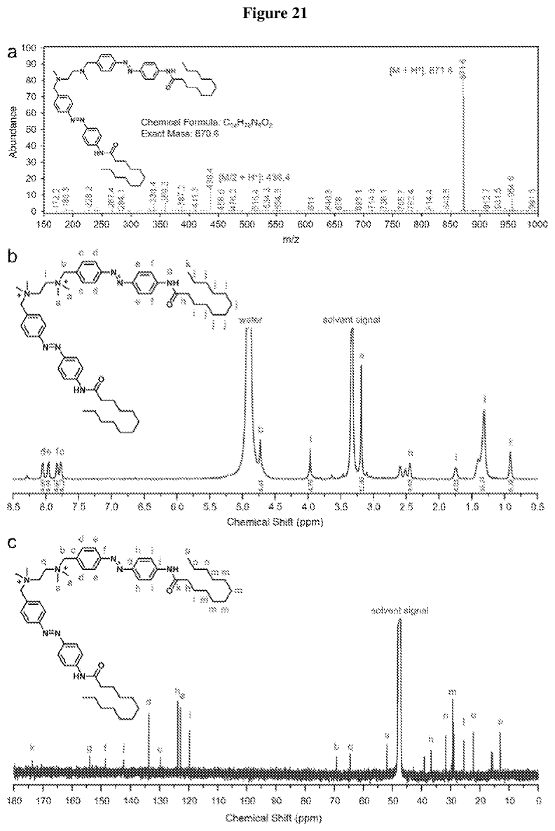

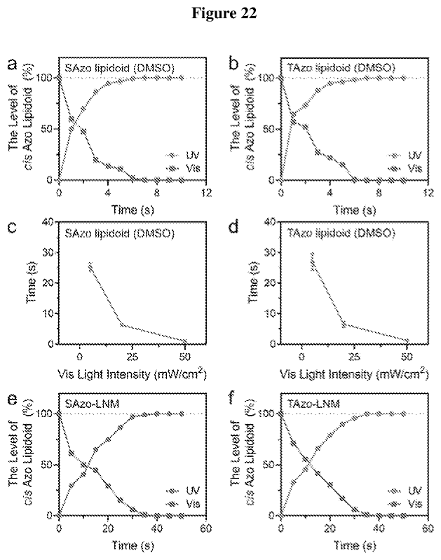

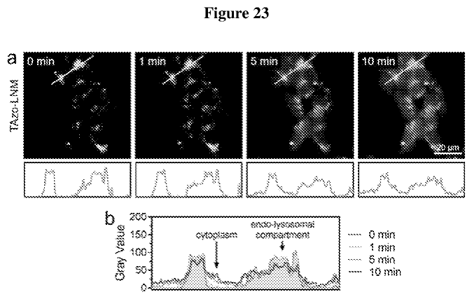

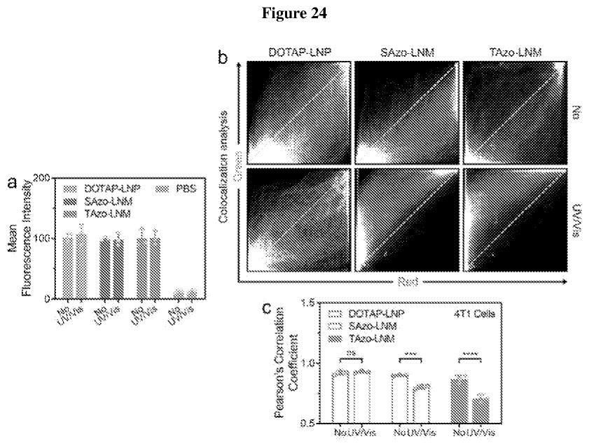

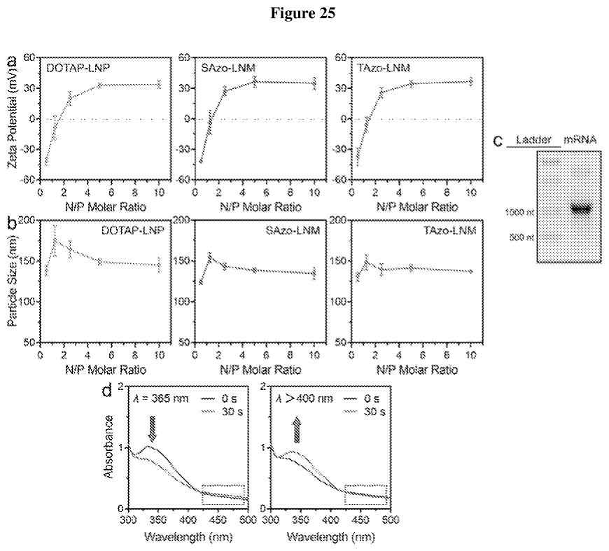

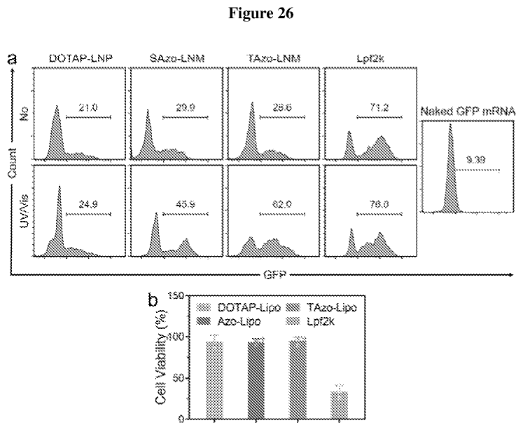

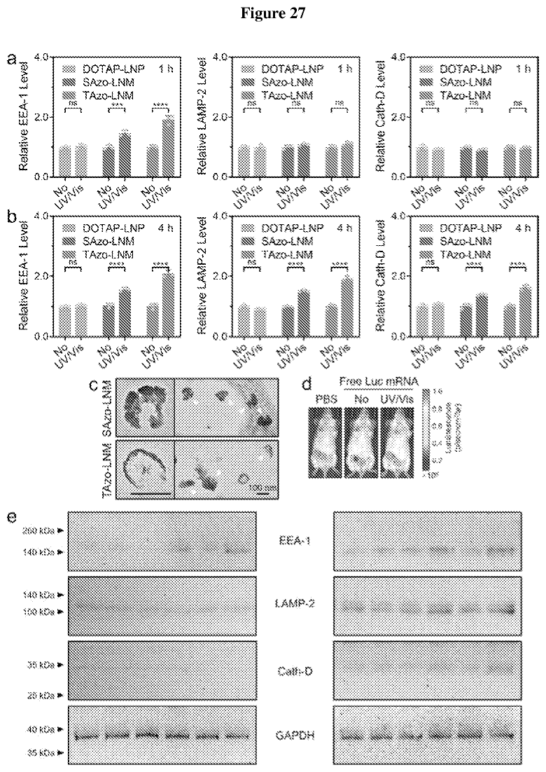

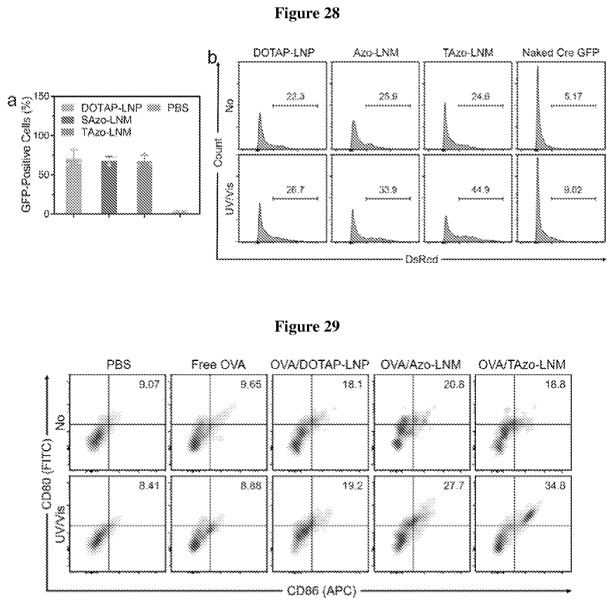

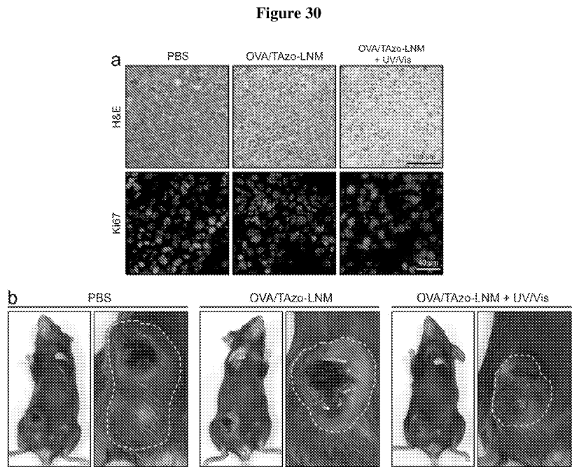

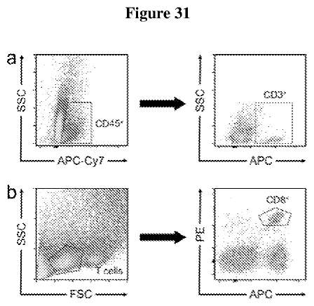

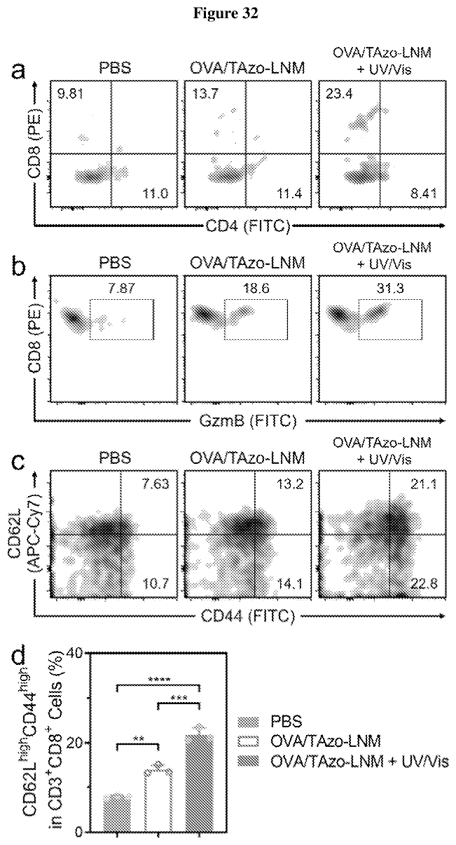

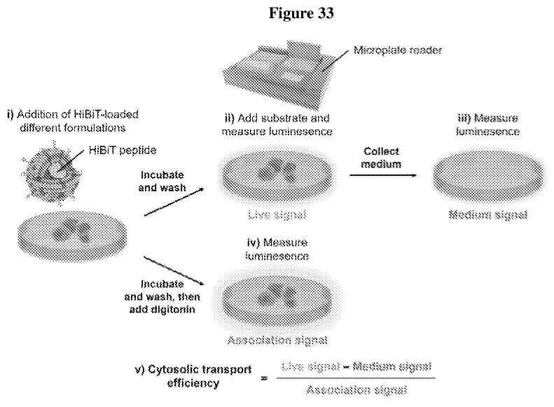

Next, the potential of LNMs to transport protein cargoes was studied. Intracellular delivery of genome-editing proteins, such as Cre recombinase (Cre) and Cas9/sgRNA ribonucleoproteins, can regulate cellular gene expression with high specificity^{a39-a41}. Thus, the capability of LNMs to transport Cre across the endo-lysosomal membranes was investigated. (−30)GFP-Cre (obtained by fusing a negatively charged GFP variant, (−30)GFP, to Cre), HeLa cells, and HeLa-DsRed cells were used in this study^{a40,a42}. (−30)GFP-Cre with SAzo-LNMs and TAzo-LNMs, respectively, were assembled and then incubated with HeLa cells. Naked (−30)GFP-Cre and DOTAP-LNP/(−30)GFP-Cre complexes were employed for comparison. The internalization of (−30)GFP-Cre by different formulations (protein, 1.5 μg mL-1; formulation, 50 μg mL-1) was first quantified by counting GFP-positive cells. Similar levels of GFP-positive cells were observed in the DOTAP-LNP, SAzo-LNM, and TAzo-LNM groups. The intracellular trafficking of (−30)GFP-Cre was then studied by visualizing the localization of (−30)GFP-Cre using CLSM imaging. A procedure similar to mRNA transport was performed. As shown in FIG. 19a, compared to DOTAP-LNPs, treatment with SAzo-LNM/(−30)GFP-Cre and TAzo-LNM/(−30)GFP-Cre complexes led to significant accumulation of GFP signal (green) in the cytoplasm and nuclei (blue) with a lower level of co-localization in endo-lysosomal compartments (red) when exposed to UV/Vis light. Furthermore, these results were confirmed with co-localization analysis (FIG. 19b) by calculating the PCC values between the fluorescence signals of (−30)GFP-Cre and LysoTracker Red. The inherent nucleus targeting capability of the Cre enabled effective accumulation of (−30)GFP-Cre in nuclei for gene recombination.

Next, to evaluate the recombination efficiency, HeLa-DsRed cells were used. These cells are genetically integrated with a LoxP-flanked STOP cassette to prevent the transcription of red fluorescent DsRed while expressing DsRed in Cre-mediated gene recombination (FIG. 19d)^{a43}. LNM/(−30)GFP-Cre complexes were incubated with cells for 4 hours, then received 10 min of UV/Vis irradiation, and incubated for another 20 hours. Following this, the percentages of DsRed-positive cells in each group was quantified to determine the recombination efficiency. The results showed that treatment of HeLa-DsRed cells with DOTAP-LNPs, SAzo-LNMs, and TAzo-LNMs resulted in significantly enhanced DsRed expression compared to naked GFP-Cre (GFP-Cre, 5.17%; DOTAP-LNPs, 22.3%; SAzo-LNMs, 25.9%; TAzo-LNMs, 24.6%; FIG. 19c. Importantly, UV/Vis light irradiation further improved the levels of DsRed-positive cells in the SAzo-LNM (33.9%) and TAzo-LNM (44.9%) groups, particularly the latter. These results indicated that light-driven LNMs also showed great potential to facilitate cytoplasm transport of protein-based biologics.

Dendritic cell (DC)-based cancer vaccines have gained notable advances in recent years44. In this therapy, DCs are typically preincubated with tumour antigens or tumour cell lysates45. These primed autologous DCs can induce T cell-dependent antitumour responses when they migrate from the administration site to the draining lymph nodes. However, only a limited number of patients benefited from DC vaccination in clinical trials. An important reason is that the tumour antigens internalized by DCs are easily degraded within lysosomal compartments, resulting in limited cross-presentation^{a46}. Considering the success of LNMs to carry protein-based biologics into the cytoplasm, whether the LNMs could transport tumour antigens into the cytoplasm to bind with MHC-I in DCs for enhanced cross-presentation was investigated.

The impact of LNMs on antigen cross-presentation was first evaluated in murine bone marrow-derived dendritic cells (BMDCs). Ovalbumin (OVA) was used as a model antigen. LNM/OVA complexes were obtained by mixing different LNMs and OVA. These complexes were then incubated with BMDCs and received UV/Vis light irradiation (FIG. 20a). The cross-presentation of OVA in BMDCs was studied by flow cytometry assay and CLSM observation. As shown in FIGS. 20b and 20c, BMDCs treated with SAzo-LNM/OVA and TAzo-LNM/OVA complexes exhibited 1.7-fold and 2.6-fold higher presentation of OVA fragments on MHC-I after UV/Vis light irradiation, respectively, compared to without irradiation, as demonstrated by staining H2kb-SIINFEKL complex. Immunofluorescence analysis confirmed this result (FIG. 20d). The highest H2kb-SIINFEKL complex signal (green) was observed on the surface of BMDCs that received TAzo-LNM/OVA complex-treatments coupled with UV/Vis light irradiation. Moreover, it was also observed that SAzo- and TAzo-LNM-treated groups showed significantly upregulated expression of the co-stimulatory markers CD80 and CD86 in BMDCs in the presence of UV/Vis irradiation, especially the latter, indicating enhanced DC maturation (FIG. 20e). These results showed that light irradiation of LNM-treatment could efficiently enhance tumour antigen cross-presentation in DCs, which is crucial to enhance T cell-dependent antitumour response in vivo.

Since TAzo-LNM showed higher efficiency than SAzo-LNM in terms of inducing both antigen cross-presentation (FIG. 20c) and DC maturation (FIG. 20e), TAzo-LNM was selected as the vehicle for DC engineering, and DC-mediated antitumour activity in an animal model was investigated. B16F10-OVA (B16F10 cells express ovalbumin) tumour-bearing mice were injected subcutaneously with PBS and BMDCs, respectively (FIG. 20a, details in Supporting Information). The BMDCs were pre-treated by TAzo-LNM/OVA complexes with or without UV/Vis light irradiation (denoted as TAzo-LNM/OVA+UV/Vis and TAzo-LNM/OVA, respectively). As shown in FIG. 20f-20h, BMDCs that were pre-treated with TAzo-LNM/OVA+UV/Vis significantly delayed the growth of B16F10-OVA tumours and extended survival time of the mice compared to other treatment groups. Similar therapeutic tendencies were observed in the tumour slices by hematoxylin and eosin (H&E) staining and cell proliferation antigen Ki-67 staining.

Then, the activation of cytotoxic CD8+ T cells within tumour tissues was analyzed since CD8+ T cells are the major effectors in antitumour immunity. According to the results, the highest CD8+ T cell infiltration was found in the tumours collected from TAzo-LNM/OVA+UV/Vis group (CD8+, 23.4%), which should be attributed to the enhanced activation of BMDCs (FIG. 20i). Moreover, CD8+ T cells in the TAzo-LNM/OVA+UV/Vis group exhibited significantly increased expression of granzyme B (GzmBhigh, 31.3%), confirming robust cytotoxic T cell activation (FIG. 20j). Next, the antitumour immune memory induced by this DC vaccine-based immunotherapy was studied. As shown in FIG. 20k, TAzo-LNM/OVA+UV/Vis-treatment resulted in the elevation in the populations of CD62LlowCD44high effector memory T cells (TEM, 22.8%) and CD62LhighCD44high central memory T cells (TCM, 21.1%) in the spleens. Memory T cells are crucial for inhibiting tumour metastasis. To further confirm the antitumour immune memory, B16F10-OVA tumour-bearing mice in TAzo-LNM/OVA and TAzo-LNM/OVA+UV/Vis groups were rechallenged with B16F10-OVA cells (1×106 cells per mouse) via intravenous (IV) injection on day 11. A PBS control group was also treated with B16F10-OVA cells via IV injection. According to the results, treatment with TAzo-LNM/OVA+UV/Vis significantly inhibited tumour metastasis to the lungs (FIG. 20l). In contrast, the mice in other groups showed obvious lung metastasis. These results suggested that LNM-based biomimetic strategy could enhance DC vaccine-based immunotherapy by directly regulating the transport of tumour antigens in DCs.

Inspired by intracellular molecular machines, an artificial lipid-based molecular machine (LNM) composed of photoisomerable Azo-based lipidoids and helper lipids was developed, which achieved enhanced cytosolic transport of exogenous biologics and certain proteins. Unlike biological molecular machines, LNM was designed to perform mechanical movements by consuming photons. Reversible isomerization of Azo-based lipidoids, caused by simultaneous UV and Vis irradiation, enabled them to produce continuous rotation-inversion movements, accompanied with stretch-shrink motions. After entering cells, LNMs interacted with the endo-lysosomal membranes, and Azo-based lipidoids acted as rotors when they received UV/Vis light irradiation, resulting in enhanced destabilization and disruption of the membranes. Thus, the cargoes could be effectively transported into the cytoplasm. In this sense, LNMs have similar functions to the biological molecular machines that can regulate intracellular cargo transport in an ideal manner. Furthermore, LNMs would be a powerful complement for the inability of biological molecular machines to transport exogenous biologics within cells.

In this work, Azo-based lipidoids were synthesized including SAzo and TAzo lipidoids to construct LNMs. It was found that TAzo-LNMs exhibited higher transmembrane transport efficiency, although both were excellent. This is because compared to the SAzo lipidoid, a larger change in the molecular dimensions of the TAzo lipidoid takes place during isomerization. The higher transmembrane efficiency of TAzo-LNMs should be the result of these dramatic changes in molecular dimensions. The capabilities of TAzo-LNMs to enhance the transfection efficiency of mRNA both in vitro and in vivo were demonstrated, as well as the Cre-mediated gene recombination at the cellular level. Moreover, the great potential of TAzo-LNMs in enhancing DC vaccine-based immunotherapy was also shown. TAzo-LNMs could carry the tumour antigens from endo-lysosomal compartments to the cytoplasm to bind with MHC-I in DCs when received light irradiation, resulting in robust cross-presentation. As a result, administration of the DCs that pre-treated with TAzo-LNM/tumour antigen complexes coupled with UV/Vis irradiation significantly inhibited the growth of tumours and lung metastasis. Considering the destructive effects of UV light, the cells were irradiated for a relatively short period of time to drive the LNMs, which just resulted in a 2.1-fold increase in cytosolic transport efficiency. The efficiency could be further improved by extending the irradiation time, especially as their design progresses to allow biocompatible near-infrared or two-photon activation47.

Our current efforts to control motions at the molecular levels may appear awkward compared with the exquisite functionalities displayed by natural systems. However, it should not be forgotten that the molecular machines produced by nature are extremely complicated systems that have evolved over time. Our synthetic systems may not be as complex as these natural machines. Despite all this, our research still provides a viable route to fabricate man-made molecular machines with biological functions via simple chemistry, which has great values in constructing soft robotics, as well as designing intelligent (next generation) drug delivery platforms.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(OXO-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

Articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

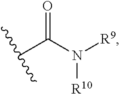

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

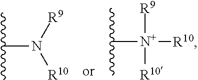

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

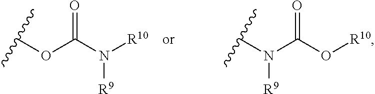

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

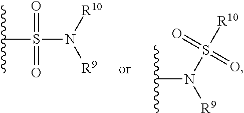

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹
wherein R⁹ represents a hydrocarbyl.

The term “thioether”, as used herein, is equivalent to an ether, wherein the oxygen is replaced with a sulfur.

The term “urea” is art-recognized and may be represented by the general formula

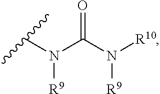

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Salt” is used herein to refer to an acid addition salt or a basic addition salt.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

The term “pharmaceutically acceptable cation” refers to an acceptable cationic counterion of an acidic functional group. Such cations are exemplified by sodium, potassium, calcium, magnesium, ammonium, tetraalkylammonium cations, and the like (see, e. g., Berge, et al., J. Pharm. Sci. 66 (1):1-79 (January 77).

“Pharmaceutically acceptable vehicle” refers to a diluent, adjuvant, excipient or carrier with which a compound of the invention is administered.

“Pharmaceutically acceptable metabolically cleavable group” refers to a group which is cleaved in vivo to yield the parent molecule of the structural formula indicated herein. Examples of metabolically cleavable groups include —COR, —COOR, —CONRR and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkylesters or (alkoxycarbonyl)oxy)alkylesters. Particularly the C₁-C₈ alkyl, C₂-C₈ alkenyl, C₂-C₈ alkynyl, aryl, C₇-C₁₂ substituted aryl, and C₇-C₁₂ arylalkyl esters of the compounds of the invention.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “subject” to which administration is contemplated includes, but is not limited to, humans (i.e., a male or female of any age group, e.g., a pediatric subject (e.g, infant, child, adolescent) or adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

An “effective amount” means the amount of a compound that, when administered to a subject for treating or preventing a disease, is sufficient to effect such treatment or prevention. The “effective amount” can vary depending on the compound, the disease and its severity, and the age, weight, etc., of the subject to be treated. A “therapeutically effective amount” refers to the effective amount for therapeutic treatment. A “prophylactically effective amount” refers to the effective amount for prophylactic treatment.

“Preventing” or “prevention” or “prophylactic treatment” refers to a reduction in risk of acquiring or developing a disease or disorder (i.e., causing at least one of the clinical symptoms of the disease not to develop in a subject not yet exposed to a disease-causing agent, or predisposed to the disease in advance of disease onset.

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be “²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radio-active isotopes tritium, i.e., ³H, and carbon-14, i.e., ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy. All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers, respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the acid- and nitro-forms of phenylnitromethane, that are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 95% by weight R-compound and at most about 5% by weight S-compound, at least about 99% by weight R-compound and at most about 1% by weight S-compound, or at least about 99.9% by weight R-compound and at most about 0.1% by weight S-compound. In certain embodiments, the weights are based upon total weight of compound.

As used herein and unless otherwise indicated, the term “enantiomerically pure S-compound” or “S-compound” refers to at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

One having ordinary skill in the art of organic synthesis will recognize that the maximum number of heteroatoms in a stable, chemically feasible heterocyclic ring, whether it is aromatic or non-aromatic, is determined by the size of the ring, the degree of unsaturation and the valence of the heteroatoms. In general, a heterocyclic ring may have one to four heteroatoms so long as the heteroaromatic ring is chemically feasible and stable.

### EXAMPLES

In order that the invention described herein may be more fully understood, the following examples are set forth. The examples described in this application are offered to illustrate the compounds, compositions, materials, device, and methods provided herein and are not to be construed in any way as limiting their scope.

### Materials

Unless otherwise noted, reagents and solvents were used as received from commercial sources without further purification. Solvents were purchased from Sigma-Aldrich. Oxone® (Oxone|r, monopersulfate), p-Toluidine, p-phenylenediamine, dodecanoic acid, thionyl chloride, trimethylamine, triethylamine, N-Bromosuccinimide, benzoyl peroxide, hydrogen peroxide (3 wt. % solution in water), phosphotungstic acid (EPTA), glucose, Quantitative Peroxide Assay Kit (Lipid), and Cell Counting Kit-8 (CCK-8) were purchased from Thermo Scientific™. Dimyristoylphosphatidylcholine (DMPC), dipalmitoylphosphatidylcholine (DPPC), distearoylphosphatidylcholine (DSPC), and 1,2-Dioleoyl-3-trimethylammoniumpropane (DOTAP) were purchased from Avanti Polar Lipids. Carbon support film 5-6 nm thick on Square 200 mesh Copper Grid was purchased from Electron Microscopy Sciences. Fluorescein isothiocyanate (FITC), Glucose Oxidase (Aspergillus niger, GOx), and Catalase (CAT) were purchased from Sigma-Aldrich. Carbonic anhydrase from bovine erythrocytes (CA) was purchased from Aladdin. Dansylamine (DNSA) and p-nitrophenyl acetate (PNPA) were purchased from TCI. Bicinchoninic acid disodium salt hydrate (BCA) was purchased from Solarbio. Dulbecco's Modified Eagle Medium (DMEM) growth medium, Fetal Bovine Serum (FBS), HEPES buffer solution (50 mM, pH 7.2), and Dulbecco's phosphate buffered saline solution (D-PBS, 10 mM, pH 7.2) were purchased from Gibco (Gibco Corporation). Trypsin-EDTA (0.25%) were purchased from Invitrogen. The 4T1 mouse breast tumor cell line was purchased from American type culture collection (ATCC). The 4T1 cell line was maintained in DMEM medium supplemented with 10% FBS, 100 units mL⁻¹ penicillin, and 100 μg mL⁻¹ streptomycin, and were kept at 37° C. in humidified 5% CO₂.

### Instruments

All ¹H nuclear magnetic resonance (¹H NMR) spectra were recorded on a Bruker AVIII 500 MHz NMR spectrometer operated in the Fourier transform mode. ESI-MS spectra was collected by Finningan LTQ. Hydrodynamic size (Dynamic light scattering measurements, DLS) and polydispersity index of nanoparticles were measured using a Zeta-PALS particle size analyzer (Brookhaven Instruments). Transmission Electron Microscopy (TEM) measurements were performed on a FEI Technai Spirit Transmission Electron Microscope.

### Synthesis of Azobenzene (Azo) Lipidoids

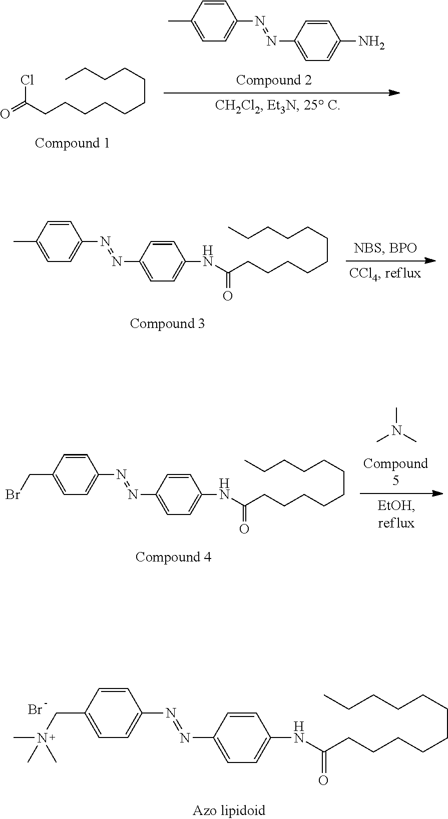

### Synthesis of Compound 1

Dodecanoic acid (2.0 g, 10 mmol) was suspended in thionyl chloride (SOCl₂, 8 mL), then the mixture was stirred at 65° C. overnight (16 h). After evaporating the excess thionyl chloride, the crude Compound 1 was obtained as a colorless oil (1.8 g, 8.23 mmol, 82.3% yield).

### Synthesis of Compound 2

A solution of Oxone® (12.3 g, 20 mmol, 2.0 eq) in water (30 mL) was added to a stirring solution of p-Toluidine (1.1 g, 10 mmol, 1.0 eq) in 20 mL of dichloromethane (CH₂Cl₂). The reaction mixture was then stirred for 6 h at room temperature in the absence of light. Subsequently, the deep green colored organic layer was separated, the aqueous solution was neutralized by addition of saturated sodium bicarbonate (NaHCO₃, 50 mL) solution and extracted with CH₂Cl₂ (3×30 mL). Anhydrous magnesium sulfate (MgSO₄) were added to the combined organic layers. After filtration, the solvent was removed in reduced pressure. The crude nitroso-compound was then dissolved in a mixture of dry CH₂Cl₂ (20 mL), p-phenylenediamine (1.1 g, 10 mmol, 1.0 eq) and glacial acetic acid (15 mL). The mixture was finally stirred overnight (16 h) in the absence of light, the solvent was then evaporated under reduced pressure and the residue was purified by column chromatography (hexane/ethyl acetate, 3/1 to 1/1, v/v) to yield Compound 2 (1.2 g, 5.7 mmol, 56.6% yield) as an orange solid. 1 TLC: R_{f}=0.61 (hexane/ethyl acetate, 3/1, v/v). ¹H NMR (CDCl₃, 500 MHz, ppm): δ 7.82 (d, J=10.0 Hz, 2H), 7.78 (d, J=10.0 Hz, 2H), 7.30 (d, J=10.0 Hz, 2H), 6.77 (d, J=10.0 Hz, 2H), 4.04 (s, 2H), 2.44 (s, 3H). ESI-MS: m/z calculated for [M+H+] C₁₃H₁₃N₃: 211.11. found: 212.27.

### Synthesis of Compound 3

Compound 2 (1.5 g, 5.8 mmol, 1.0 eq) was dissolved in dry CH₂Cl₂ (30 mL) and triethylamine (Et₃N, 1.2 g, 11.6 mmol, 2.0 eq) was added to the solution. Compound 1 (1.9 g, 8.8 mmol, 1.5 eq) was dissolved in dry CH₂Cl₂ (10 mL) and then added dropwise to the above solution of compound 2. After stirring overnight (16 h) at 25° C., the solvent was removed under reduced pressure to yield a brown solid. The residue was purified by column chromatography (hexane/ethyl acetate, 5/1 to 1/1, v/v) to yield Compound 3 (2.1 g, 5.3 mmol, 92.1% yield) as an orange solid. TLC: Rf=0.47 (hexane/ethyl acetate, 5/1, v/v). ¹H NMR (CDCl₃, 500 MHz, ppm): δ 7.93 (d, J=10.0 Hz, 2H), 7.83 (d, J=10.0 Hz, 2H), 7.70 (d, J=10.0 Hz, 2H), 7.33 (d, J=10.0 Hz, 2H), 7.27 (s, 1H), 2.46 (s, 3H), 2.43-2.38 (m, 2H), 1.81-1.75 (m, 2H), 1.42-1.29 (m, 16H), 0.91 (t, J=5.0 Hz, 3H). ESI-MS: m/z calculated for [M+H⁺] C₂₅H₃₅N₃O: 394.28. found: 394.36.

### Synthesis of Compound 4

Compound 3 (0.5 g, 1.3 mmol, 1.0 eq), N-Bromosuccinimide (NBS, 0.35 g, 1.9 mmol, 1.5 eq), and benzoyl peroxide (BPO, 15 mg) were dissolved in carbon tetrachloride (CCl₄, 15 mL) under an argon atmosphere, then the reaction mixture was heated at reflux for 48 h. The Compound 4 and Compound 3 have the same Rf value based on TLC, which was confirmed by the ¹H NMR spectrum. When ¹H NMR spectroscopy showed that the reaction was almost completed, the mixture was cooled to 0° C. and filtered under reduced pressure to yield a yellow solid, which was washed with cold diethyl ether (3×20 mL). The unpurified Compound 4 was obtained as a yellow solid (0.5 g, 1.1 mmol, 81.7% yield).

### Synthesis of Azo Lipidoids

A solution of unpurified Compound 4 (0.5 g, 1.1 mmol, 1.0 eq) in ethanol (10 mL) and trimethylamine (Me₃N, Compound 5, 45% in water, 3.0 mL, >10 eq) was stirred at reflux temperature for 24 h under an argon atmosphere. The solution was concentrated under reduced pressure. The residue was diluted with ice diethyl ether/CH₂Cl₂ (5/1, v/v, 15.0 mL). The orange precipitate was collected by filtration and washed with ice diethyl ether/CH₂Cl₂ (5/1, v/v) to remove unreacted Compound 4 and Compound 5. The desired Azo lipidoid was obtained as an orange solid (0.3 g, 0.7 mmol, 60.3% yield).

¹H NMR (CD₃OD, 500 MHz, ppm): δ 8.04 (d, J=5.0 Hz, 2H), 7.96 (d, J=10.0 Hz, 2H), 7.82 (d, J=5.0 Hz, 2H), 7.76 (d, J=5.0 Hz, 2H), 4.64 (s, 2H), 3.19 (s, 9H), 2.44 (t, J=5.0 Hz, 2H), 1.76-1.72 (m, 2H), 1.41-1.32 (m, 16H), 0.91 (t, J=5.0 Hz, 3H). ¹³C NMR (CD₃OD, ppm): δ 173.7, 153.9, 148.6, 142.4, 133.8, 129.7, 123.9, 122.8, 119.7, 68.6, 52.0, 36.7, 31.7, 29.3, 29.2, 29.1, 28.9, 25.4, 22.3, 13.1. ESI-MS: m/z calculated for [M+H⁺] C₂₈H₄₃N₄O: 452.34. found: 452.45.

### The Photoisomerization of Azo Lipidoids

Azo lipidoid was prepared as 50 μM solution in anhydrous dimethyl sulfoxide (DMSO). The sample was irradiated with UV (365 nm for 6 s) and Vis light (>400 nm for 6 s), respectively. The power densities for 365 and >400 nm was 5.40 and 20 mW/cm², respectively.

### Preparation of Lipidoid Artificial Compartments

The lipidoid artificial compartments were prepared via thin lipid film hydration methods. Azo lipidoid, distearoylphosphatidylcholine (DSPC), dimyristoylphosphatidylcholine (DMPC), dimyristoylphosphatidylcholine (DPPC), 1,2-dioleoyloxy-3-trimethylammonium propane chloride (DOTAP), cholesterol (CH), and 1,2-Distearoyl-sn-glycero-3-phosphoethanolamine-N-methoxy-poly(ethylene glycol) (mPEG-DSPE, 5000) were dissolved in chloroform to make a stock solution, respectively.

Preparation of Azo Lipidoid Compartment with Various Helper Lipids.

Briefly, the lipid mixture comprising Azo lipidoid, helper lipid (DSPC or DMPC or DPPC), DOTAP, CH, and mPEG-DSPE in certain molar ratio was first dissolved in a chloroform/methanol mixture (4:1, v/v). After evaporation of the organic solvent, the lipid film was hydrated with HEPES buffer at 50° C. for 30 min. Following hydration, the solution was repeatedly extruded through a 200 nm polycarbonate membranes filter to obtain the azo lipidoid compartment. Compartments with a series of Azo lipidoid percentage (from 0% to 20% molar ratio) were prepared below.
0% Azo lipidoid: the molar ratio of Azo lipidoid:helper lipid:DOTAP:CH:mPEG-DSPE was 0:65:15:15:5.
2.5% Azo lipidoid: the molar ratio of Azo lipidoid:helper lipid:DOTAP:CH:mPEG-DSPE was 2.5:65:12.5:15:5.
5% Azo lipidoid: the molar ratio of Azo lipidoid:helper lipid:DOTAP:CH:mPEG-DSPE was 5:65:10:15:5.
10% Azo lipidoid: the molar ratio of Azo lipidoid:helper lipid:DOTAP:CH:mPEG-DSPE was 10:65:5:15:5.
15% Azo lipidoid: the molar ratio of Azo lipidoid:helper lipid:DOTAP:CH:mPEG-DSPE was 15:65:0:15:5
20% Azo lipidoid: the molar ratio of Azo lipidoid:helper lipid:DOTAP:CH:mPEG-DSPE was 20:60:0:15:5.

### Preparation of CA, CAT and GOx-Encapsulated Lipidoid Artificial Compartments

Enzyme (CA or CAT or GOx)-encapsulated lipidoid artificial compartments were prepared with DSPC as the helper lipid using a similar method described above. In brief, the lipid mixture (10 mg) was dissolved in a chloroform/methanol mixture (4:1, v/v). After evaporation of the organic solvents, the lipid film was hydrated with 2 mL of HEPES buffer containing 1 mg enzyme (CA or CAT or GOx)under ultrasound for 30 min (non-continuous). Subsequently, the solution was passed through a size exclusion column (Sepharose 6B) to remove the unencapsulated enzymes. The fractions of enzyme-encapsulated lipidoid artificial compartments were collected, concentrated by centrifugal filtration (molecular weight cut off, MW 100 kDa), and then washed by HEPES buffer (50 mM, pH 7.2, 150 mM NaCl). The solution was then dialyzed against HEPES buffer (50 mM, pH 7.2) and stored at 4° C. for further use. The concentration of the enzyme was determined by the bicinchoninic acid (BCA) assay.

### Characterization of Lipidoid Artificial Compartments

### TEM

TEM characterization was performed with a FEI Technai Spirit Transmission Electron Microscope. To prepare the TEM sample, 10 μL sample solution was dropped onto a carbon-coated copper grid for 10~15 min and blotted with filter paper to remove excess liquid. Then the sample was negatively stained with phosphotungstic acid (EPTA) (5~10 μL) for 1-2 min, blotted again and air-dried before analysis on TEM.

### DLS Measurements

The hydrodynamic size of different lipidoid artificial compartments was evaluated in HEPES buffer at 37° C.

### The Photoisomerization of Azo Lipidoids in Lipidoid Artificial Compartments

The photoisomerization properties of different lipidoid artificial compartments (50 μM, using Azo lipidoid as standard) were monitored in HEPES buffer (50 mM, pH 7.2) at 37° C. The sample was irradiated with UV (365 nm for 30 s) and Visible light (>400 nm for 30 s), respectively. The power densities for 365 and >400 nm was 5.40 and 20 mW/cm², respectively.

### Quantification of CA Adsorption on the Surface of Artificial Compartments

CA adsorption was quantified using the following method. The empty artificial compartments (1 mg/mL) were incubated with CA (0.5 mg/mL) at 37° C. for 30 min. After incubation, the samples were filtered and washed 3 times with HEPES buffer (50 mM, pH 7.2, 150 mM NaCl) using centrifugal filtration (molecular weight cut off, MW 100 kDa) to remove unabsorbed CA (MW, ~30 kDa). After reconstituting with 50 μL of HEPES buffer, the amount of absorbed CA in each sample was determined by the bicinchoninic acid (BCA) assay using bovine serum albumin (BSA) as the standard.

### Effect of Percentage of Azo Lipidoids on the Compartment Stability Under Irradiation

The CA-encapsulated lipidoid artificial compartments (0.1 mg/mL, CA as the standard) with various molar percentage (0, 2.5, 5, 10, 20%) of Azo lipidoids were loaded into the dialysis bag (molecular weight cut off, MW 100 kDa), and incubated at 37° C. for 15 min under UV/Vis light irradiation. The amount of CA leaked out from the lipidoid artificial compartments in each sample was determined by the bicinchoninic acid (BCA) assay using bovine serum albumin (BSA) as the standard.

The release of CA was also evaluated using gel electrophoresis. CA was first labeled FITC according to literature. Briefly, the CA was dissolved in sodium carbonate buffer (5 mg/mL, pH 8.5). FITC was dissolved with DMSO to make 1% (m/v) stock solution. FITC stock solution was added into CA solution at a molar ratio of 5/1 (Dye/Enzyme, n/n). The reactions were kept at 4° C. for overnight. As-formed samples were dialyzed against HEPES buffer to remove the unconjugated dye. FITC-CA encapsulated lipidoid compartment with various molar percentage (0, 2.5, 5, 10, 15 and 20%) of Azo lipidoids were incubated at 37° C. for 15 min under UV/Vis light irradiation. Then the solution was run through electrophoresis to detect the released FITC-CA and encapsulated CA.

### Measuring CA Enzyme Activity

The apparent Kinetic parameter (Km) values were determined by Lineweaver-Burk plotting method. In 150 μL of free CA or encapsulated CA solution (1 μM of CA), different amount of substrate p-nitrophenyl acetate (PNPA) were added to obtain a final PNPA concentrations of 0.625, 1.25, 2.5, 5.0, 10.0, 20.0 mM. The absorbance of the solution at 405 nm was continuously recorded using a UV-Vis plate reader. The Kₘ was obtained through Lineweaver-Burk plotting method.

Bidirectional regulation of the enzyme activity of CA (1 μM) in different formulations was performed in HEPES buffer at 37° C. using PNPA (1.0 mM) as substrate. The CA activity assay, both in free form and in compartment, was performed according to previously published procedure.

1 The enzyme activity assay is started by mixing 20 μL of PNPA (HEPES buffer, 1.0 mM) with 150 μL of different CA-encapsulated lipidoid artificial compartments (1 μM, CA as the standard) in HEPES buffer (50 mM, pH 7.2) in 37° C. The absorbance of the solution at 405 nm was recorded immediately, as that at time 0 s.

2 The sample solution was further incubated for 60 s without light irradiation. The absorbance of the solution at 405 nm was recorded immediately, as that at time 60 s.

3 The sample solution was then irradiated with UV/Vis light for 60 s, and then the absorbance of the solution at 405 nm was recorded, as that at time 120 s.

4 Repeat Step 2) and 3) to get the absorbances of the solution at 405 nm after incubation at 180s (no irradiation), 240s (irradiation), 300s (no irradiation), 360s (irradiation).

5 The absorbance at 405 nm at each time point was plotted against the time point, and the relative enzyme activity was calculated using the slope of the curve.

### Measuring the CAT Enzyme Activity

The CAT enzyme activity was determined by quantifying the amount of oxygen generated when incubating the enzyme with H₂O₂ (250 μM). A portable dissolved oxygen meter (RCYAGO Smart Bluetooth Dissolved Oxygen Meter) was used to measure the oxygen level.⁵ The bidirectional regulation of the CAT enzyme activity, both in free form and in compartment, was performed using the same procedure as CA study described above, except using H₂O₂ as the substrate.

### Measuring GOx Enzyme Activity

The GOx enzyme activity was determined by the generation of H₂O₂ using glucose (1 mg/mL) as the substrate. The level of H₂O₂ in glucose solution was monitored using a Quantitative Peroxide Assay Kit (Lipid). The bidirectional regulation of the GOx enzyme activity, both in free form and in the compartment, was performed using the same procedure as CA study described above, except using glucose as the substrate.

### Cell Cytotoxicity Using CCK-8 Assay

In vitro cytotoxicity was assessed by the standard CCK-8 assay. To test the cytotoxicity of DSPC-Azo@GOx with or without UV/Vis light irradiation, 4T1 tumor cells were seeded in the basolateral chamber of a 24-well transwell plate at a density of 105 cells per well and cultured in 5% CO₂ at 37° C. for overnight (16 h). Then, DSPC-Azo@GOx was added to the upper chamber. Then, the samples were irradiated using the UV/Vis light (irradiated for 5 min every 8 hours). After three irradiation, the cell viability was measured using CCK-8 kit according to the manufacturer's instruction.

### Synthesis of SAzo and TAzo Lipidoids

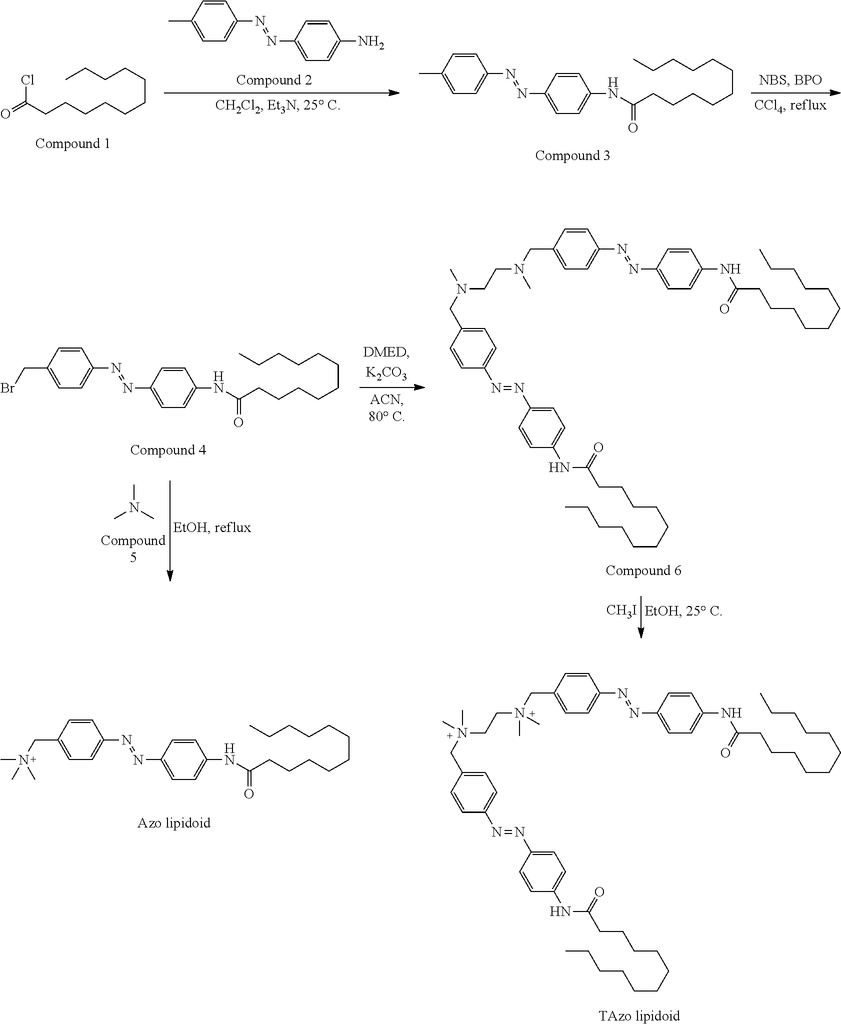

### Synthesis of Compound 1

Dodecanoic acid (2.0 g, 10 mmol) was suspended in thionyl chloride (SOCl2, 8 mL), then the mixture was stirred at 65° C. overnight (16 hours). After evaporating the excess thionyl chloride, the crude Compound 1 was obtained as a colorless oil (1.7 g, 7.82 mmol, 7.82% yield).

### Synthesis of Compound 2

To a stirring solution of p-Toluidine (1.1 g, 10 mmol, 1.0 eq) in 20 mL of dichloromethane (CH₂Cl₂) was added a solution of Oxone® (12.3 g, 20 mmol, 2.0 eq) in water (30 mL). The reaction mixture was then stirred for 6 hours at room temperature in the absence of light. Subsequently, the deep green coloured organic layer was separated, the aqueous solution was neutralized by the addition of saturated solution of sodium bicarbonate (NaHCO₃, 50 mL) and extracted with CH₂Cl₂ (3×30 mL). The combined organic layers were tried with anhydrous magnesium sulfate (MgSO₄), filtered off and the solvent was removed in reduced pressure. The crude nitroso-compound was then dissolved in a mixture of dry CH₂Cl₂ (20 mL). To this solution p-phenylenediamine (1.1 g, 10 mmol, 1.0 eq) and glacial acetic acid (15 mL) was added. The mixture was finally stirred overnight (16 hours) in the absence of light, the solvent was then evaporated under reduced pressure and the residue was purified by column chromatography (hexane/ethyl acetate, 3/1 to 1/1, v/v) to yield Compound 2 (1.4 g, 6.8 mmol, 67.9% yield) as an orange solid. TLC: R_{f}=0.59 (hexane/ethyl acetate, 3/1, v/v). ¹H NMR (CDCl3, 500 MHz, ppm): δ 7.82 (d, J=10.0 Hz, 2H), 7.78 (d, J=10.0 Hz, 2H), 7.30 (d, J=10.0 Hz, 2H), 6.77 (d, J=10.0 Hz, 2H), 4.04 (s, 2H), 2.44 (s, 3H). ESI-MS: m/z calculated for [M+H+] C₁₃H₁₃N₃: 211.11. found: 212.23.

### Synthesis of Compound 3

Compound 2 (1.5 g, 5.8 mmol, 1.0 eq) was dissolved in dry CH₂Cl₂ (30 mL) and triethylamine (Et₃N, 1.2 g, 11.6 mmol, 2.0 eq) was added to the solution. Compound 1 (1.9 g, 8.8 mmol, 1.5 eq) was dissolved in dry CH2Cl2 (10 mL) and then it was added dropwise to the above solution of compound 2. After stirring overnight (16 hours) at 25° C., the solvent was removed under reduced pressure to yield a brown solid. The residue was purified by column chromatography (hexane/ethyl acetate, 5/1 to 1/1, v/v) to yield Compound 3 (1.9 g, 4.8 mmol, 82.9% yield) as an orange solid. TLC: R_{f}=0.45 (hexane/ethyl acetate, 5/1, v/v). ¹H NMR (CDCl3, 500 MHz, ppm): δ 7.92 (d, J=10.0 Hz, 2H), 7.83 (d, J=10.0 Hz, 2H), 7.71 (d, J=10.0 Hz, 2H), 7.38 (s, 1H), 7.32 (d, J=10.0 Hz, 2H), 2.45 (s, 3H), 2.43-2.36 (m, 2H), 1.81-1.75 (m, 2H), 1.42-1.28 (m, 16H), 0.91 (t, J=5.0 Hz, 3H). ESI-MS: m/z calculated for [M+H+] C25H35N3O: 394.28. found: 394.38.

### Synthesis of Compound 4

Compound 3 (0.5 g, 1.3 mmol, 1.0 eq), N-Bromosuccinimide (NBS, 0.35 g, 1.9 mmol, 1.5 eq), and benzoyl peroxide (BPO, 15 mg) were dissolved in carbon tetrachloride (CCl4, 15 mL) under an argon atmosphere, then the reaction mixture was heated at reflux for 48 hours. The Compound 4 and Compound 3 have the same Rr value based on TLC, which was confirmed by the ¹H NMR spectrum. When ¹H NMR spectroscopy showed that the reaction was almost completed, the mixture was cooled to 0° C. and filtered under reduced pressure to yield a yellow solid, which was washed with cold diethyl ether (3×20 mL). Finally, the unpurified Compound 4 was obtained as a yellow solid (0.3 g).

### Synthesis of SAzo Lipidoid

A solution of unpurified Compound 4 (0.5 g, 1.1 mmol, 1.0 eq) in ethanol (10 mL) and trimethylamine (Me₃N, Compound 5, 45% in water, 3.0 mL, >10 eq) was stirred at reflux temperature for 24 hours under an argon atmosphere. The solution was concentrated under reduced pressure. The residue was diluted with ice diethyl ether/CH₂Cl₂ (5/1, v/v, 15.0 mL), the orange precipitate was collected by filtration and washed with ice diethyl ether/CH₂Cl₂ (5/1, v/v) to remove unreacted Compound 4 and Compound 5, and then the desired SAzo lipidoid was obtained as an orange solid (0.3 g, 0.8 mmol, 66.3% yield). ¹H NMR (CD₃OD, 500 MHz, ppm): δ 8.05 (d, J=5.0 Hz, 2H), 7.96 (d, J=10.0 Hz, 2H), 7.83 (d, J=5.0 Hz, 2H), 7.77 (d, J=5.0 Hz, 2H), 4.64 (s, 2H), 3.19 (s, 9H), 2.44 (t, J=5.0 Hz, 2H), 1.76-1.73 (m, 2H), 1.40-1.30 (m, 16H), 0.91 (t, J=5.0 Hz, 3H). ¹³C NMR (CD₃OD, ppm): δ 173.8, 153.9, 148.6, 142.4, 133.8, 129.7, 123.7, 122.8, 119.6, 68.6, 52.0, 36.7, 31.7, 29.3, 29.2, 29.1, 28.9, 25.4, 22.3, 13.1. ESI-MS: m/z calculated for [M+] C₂₈H₄₃N₄O: 451.34. found: 451.70.

### Synthesis of TAzo Lipidoid

TAzo lipidoid was synthesized via a one-pot method. Unpurified Compound 4 (1.0 g, 2.1 mmol, 1.0 eq), potassium carbonate (K₂CO₃) and N,N′-dimethylethylenediamine (DMED, 18.5 mg, 0.2 mmol, 0.1 eq) were dissolved in anhydrous acetonitrile (40 mL) and stirred at 60° C. for 30 hours under an argon atmosphere. TLC: R_{f}=0.60 (hexane/ethyl acetate, 5/1, v/v). The Compound 6 was confirmed by HPLC-ESI-MS. The solution was concentrated under reduced pressure and dissolved in 40 mL of ethanol. Iodomethane (0.6 g, 4.2 mmol, 2 eq) was added into the above solution and stirred at 25° C. for 24 hours under an argon atmosphere. The solution was then concentrated under reduced pressure. The residue was diluted with ice diethyl ether/CH₂Cl₂ (5/1, v/v, 30.0 mL), the red-brown precipitate was collected by filtration and washed with ice diethyl ether/CH₂Cl₂ (5/1, v/v, 3×20 mL). The desired TAzo lipidoid was obtained as a red-brown colored solid (0.10 g, 0.11 mmol, 10.5% yield). 1H NMR (CD₃OD, 500 MHz, ppm): δ 8.04 (d, J=5.0 Hz, 4H), 7.96 (d, J=10.0 Hz, 4H), 7.82 (d, J=5.0 Hz, 4H), 7.76 (d, J=5.0 Hz, 4H), 4.73 (s, 2H), 3.97 (s, 4H), 3.18 (s, 12H), 2.45 (t, J=5.0 Hz, 4H), 1.74 (m, 4H), 1.40-1.31 (m, 32H), 0.91 (t, J=5.0 Hz, 6H). 13C NMR (CD₃OD, ppm): δ 173.7, 154.0, 148.6, 142.4, 133.7, 129.7, 123.7, 122.8, 119.7, 69.2, 64.4, 51.9, 36.7, 31.6, 29.3, 29.2, 29.0, 28.9, 25.4, 22.3, 13.0. ESI-MS: m/z calculated for [M/2+] C₅₆H₈₄N₈O₂: 450.34. found: 450.45.

### The Photoisomerization of SAzo and TAzo Lipidoids

SAzo lipidoid was prepared as 30 μM solution in anhydrous dimethyl sulfoxide (DMSO). TAzo lipidoid was prepared as 15 μM solution in DMSO. These samples were irradiated with UV (365 nm for 6 s) and Vis light (>400 nm for 6 s), respectively. The power densities for 365 and >400 nm are 5.40 and 20 mW/cm², respectively.

### Preparation of LNM Formulations

### Preparation of SAzo-LNMs and TAzo-LNMs

Two different LNMs were prepared by co-assembling of SAzo or TAzo lipidoids with 1,2-dioleoyl-sn-glycero-3-phosphoethanolamine (DOPE), cholesterol (Chol), and 1,2-dimyristoyl-rac-glycero-3-methoxypolyethylene glycol-2000 (DMG-PEG) (molar ratio of 30:30:36.5:3.5, Azo units as standard) using the ethanol dilution method.

Preparation of mRNA-Loaded LNMs

All lipids with specified molar ratios were dissolved in ethanol and mRNA was dissolved in 10 mM citrate buffer (pH 4.0). The two solutions were rapidly mixed at an aqueous to ethanol ratio of 3/1 by volume (3/1, aq./ethanol) to satisfy a final weight ratio of 50/1 (total lipids/mRNA, N/P molar ratio of 2.5), then incubated for 10 min at room temperature and dialyzed at 4° C. against PBS.

### Characterization of SAzo-LNMs and TAzo-LNMs

### TEM Measurements

To prepare the TEM sample, 10 μL sample solution was dropped onto a carbon-coated copper grid for 10~15 min and blotted with filter paper to remove excess liquid. Then the sample was negatively stained with phosphotungstic acid (EPTA) (5~10 μL) for 1-2 min, blotted again and air-dried before analysis on TEM.

### DLS Measurements

The hydrodynamic size of different formulations was evaluated in PBS at 37° C.

SAzo-LNMs and TAzo-LNMs Facilitate Cytosolic Transport of mRNA

Confocal laser scanning microscope (CLSM) observation and flow cytometry and were used to study the capability of SAzo-LNMs and TAzo-LNMs to reach the cytoplasm from endo-lysosomal compartments in HeLa cells. Different NBD-labeled formulations were prepared by using L-α-phosphatidylethanolamine-N-(4-nitro-benzo-2-oxa-1,3-diazole) (PE-NBD, a fluorescent lipid).

For CLSM observation (fluorescence imaging), HeLa cells were seeded in confocal dishes and allowed to grow until approximately 80% confluence. 50 μg of NBD-labeled different formulations (green) was added and cultured with cells for 1 hour. The cells were then received UV/Vis light irradiation for 10 min. After another 30 min incubation, the cells were washed with PBS three times, and the endo-lysosomal compartments were stained with LysoTracker Red. Then, the cells were fixed using 4% paraformaldehyde, and the nucleus was stained with DAPI (blue).

For flow cytometry assays, HeLa cells (5×10⁴ cells per well) were seeded in a 12-well plate and incubated overnight for cell attachment. Different mRNA-loaded formulations (GFP mRNA, 1 μg mL-1) were incubated with cells for 4 hours, then received irradiation for 10 min. After another 20 h incubation, the cells were washed with PBS three times, followed by digesting using 0.25% trypsin for flow cytometry analysis.

### The Mechanism of Cytosolic Transport

Flow cytometry and western blots were used to study the mechanism of cytosolic transport.

For flow cytometry assays, HeLa cells (5×10⁴ cells per well) were seeded in a 12-well plate and incubated overnight for cell attachment. 50 μg of different formulations were incubated with cells for 1 hour, then received irradiation for 10 min. After another 30 min incubation, the cells were washed with PBS three times and stained with LysoSensor Green, followed by digesting using 0.25% trypsin for flow cytometry analysis.

For western blots, HeLa cells (1×10⁵ cells per well) were seeded in a 6-well plate and incubated overnight for cell attachment. 50 μg of different formulations were incubated with cells for 1 hour (and 4 hours), then received irradiation for 10 min. After another 30 min incubation, the cells were washed with PBS three times and lysed in 0.5 ml of lysis buffer (PBS containing 1% Triton X-100 and 1 mM PMSF) at 4° C. for 30 min. Equal amounts protein loadings were separated by SDS-PAGE and electrophoretically transferred to a PVDF membrane. Nonspecific binding sites were blocked with 5% nonfat milk in PBS and 0.05% Tween 20 (PBST) for 2 h at room temperature. Membranes were incubated in the primary antibodies at appropriate concentrations for 24 h at 4° C. overnight, followed by three times rinse with PBST buffer for 15 min. The membranes were then incubated in the secondary antibodies (1:10000) for 60 min at room temperature. The membranes were detected and analyzed using a computerized chemiluminescent imaging system.

### Quantification of Cytosolic Transport Efficiency

HeLa cells (4×10⁵ cells per well) were seeded in a 6-well plate and incubated overnight for cell attachment. The next day, cells were transfected with 0.5 μg of the LgBiT expression vector. After overnight incubation, cells were reseeded in a white 96-well plate and incubated overnight for cell attachment.

The SLEEQ assay was then performed according to the following procedure.

(i) HiBiT-loaded different formulations were added to the cells.

(ii) After incubation for 4 hours, the cells received UV/Vis light irradiation for 10 min. Excess HiBiT was removed, followed by another 30 min incubation. Addition of substrate allowed the measurement of the luminescence from complemented HiBiT/LgBiT in cytoplasm.

(iii) To take into account any luminescent signal from excreted LgBiT, the cell culture medium was collected and measured.

(iv) To determine the total association, digitonin was added to the HiBiT-treated cells to permeabilize cell membranes. This enabled any HiBiT trapped in endo-lysosomal compartments to complement with LgBiT.

(v) The cytosolic transport efficiency was calculated. The luminescence was measured using microplate reader.

SAzo-LNMs and TAzo-LNMs Facilitate Cytosolic Transport of mRNA In Vivo

All animal procedures were performed with ethical compliance and approval by the Institutional Animal Care and Use Committee at the Tufts University. The female Balb/c mice at 6~8-week old (Charles River) were used in this study. Balb/c mice were irradiated with UV/Vis light for 15 min at 1 h and 2 h after subcutaneous (SC) injection of Luc mRNA-loaded LNMs (0.25 mg kg-1 Luc mRNA, 5 μg per mouse), respectively. The power densities for 365 and >400 nm are 30 and 120 mW/cm2, respectively. IVIS images were taken at 5 h post-injection.

### SAzo-LNMs and TAzo-LNMs Facilitate Cytosolic Transport of Cre Proteins

Confocal laser scanning microscope (CLSM) observation and flow cytometry and were used to study the capability of SAzo-LNMs and TAzo-LNMs to transport Cre proteins from endo-lysosomal compartments to the cytoplasm. (−30)GFP-Cre (obtained by fusing a negatively charged GFP variant, (−30)GFP, to Cre), HeLa cells, and HeLa-DsRed cells were used in this study.

For CLSM observation (fluorescence imaging), HeLa cells were seeded in confocal dishes and allowed to grow until approximately 80% confluence. DOTAP-LNP/(−30)GFP-Cre, SAzo-LNM/(−30)GFP-Cre, and TAzo-LNM/(−30)GFP-Cre (green, protein, 1.5 μg mL-1; formulation, 50 μg mL-1) were added and cultured with cells for 1 hour. The cells were then received UV/Vis light irradiation for 10 min. After further incubation, the cells were washed with PBS three times, and the endo-lysosomal compartments were stained with LysoTracker Red. Then, the cells were fixed using 4% paraformaldehyde, and the nucleus was stained with DAPI (blue).

For flow cytometry assays, HeLa-DsRed cells (5×10⁴ cells per well) were seeded in a 12-well plate and incubated overnight for cell attachment. DOTAP-LNP/(−30)GFP-Cre, SAzo-LNM/(−30)GFP-Cre, and TAzo-LNM/(−30)GFP-Cre (1.5 μg mL-1 protein, green) were incubated with cells for 4 hours, then received irradiation for 10 min. After another 20 h incubation, the cells were washed with PBS three times, followed by digesting using 0.25% trypsin for flow cytometry analysis.

### TAzo-LNMs Facilitate Cross-Presentation of Tumour Antigens

All animal procedures were performed with ethical compliance and approval by the Institutional Animal Care and Use Committee at the Tufts University. The female C57BL/6 mice (Charles River) at 6~8-week old were used in this study. Bone marrow-derived dendritic cells (BMDCs) were generated from the bone marrow of C57BL/6 mice.

The BMDCs were incubated with LNM/OVA complexes (OVA, 25 μg mL⁻¹; formulation, 250 μg mL⁻¹) for 6 hours, then received UV/Vis light irradiation for 10 min. After another 18 h incubation, the BMDCs are collected and stained with fluorescent conjugated antibodies for flow cytometry analysis (e.g., PE-anti-mouse CD11C antibody, APC-anti-mouse H2kb-SIINFEKL antibody, FITC-anti-mouse CD80 antibody, and APC-anti-mouse CD86 antibody).

### Antitumor Efficacy in a Mouse Model of Melanoma

B16F10 tumour cells expressing ovalbumin (B16F10-OVA) were used in this study. B16F10-OVA tumour-bearing C57BL/6 mice were randomly divided into three groups (n=6) after six days of inoculation. The mice were then subcutaneously injected with BMDCs (1×10⁶ for per mouse) every five days for three times. The BMDCs were pre-treated by TAzo-LNM/OVA complexes with and without UV/Vis light irradiation. The tumor volume (V) was calculated according to the formula: V=L×W²/2, where L and W were the longest and shortest diameter (mm) of the tumor, respectively. The mice were euthanized when exhibiting signs of impaired health or when the volume of the tumor exceeded 1.5 cm³.

For flow cytometry assays, freshly harvested tumor tissues were digested with collagenase IV and made into single-cell suspensions according to the manufacturer's instructions. After that, cells were collected and diluted to 1×10⁷ cells mL⁻¹. 100 μL cells were stained by adding a cocktail of fluorescent conjugated antibodies on the ice for flow cytometry analysis (e.g., APC/Cy7 anti-mouse CD45 antibody, APC anti-CD3 antibody, PE anti-mouse CD8 antibody, FITC-anti-mouse CD4 antibody, and FITC-anti-mouse GramB antibody).

### Inhabitation of Tumour Metastasis to the Lung

B16F10-OVA tumour-bearing mice in TAzo-LNM/OVA and TAzo-LNM/OVA+UV/Vis groups were rechallenged with B16F10-OVA cells (1×10⁶ cells per mouse) via intravenous (IV) injection on day 11. A PBS control group was also treated with B16F10-OVA cells (1×10⁶ cells per mouse) via IV injection. On day 25, all mice were euthanized and the lungs were collected for photograph.

### Statistical Analysis

Data were expressed as mean±SD. All data were analyzed using Graphpad Prism (8.0) software.

### REFERENCES CITED

(1) Wang, Y.; Han, P.; Xu, H.; Wang, Z.; Zhang, X.; Kabanov, A. V., Photocontrolled self-assembly and disassembly of block ionomer complex vesicles: a facile approach toward supramolecular polymer nanocontainers. Langmuir 2010, 26 (2), 709-715.
(2) Moss, R. A.; Jiang, W., Thermal modulation of photoisomerization in double-azobenzene-chain liposomes. Langmuir 1997, 13 (17), 4498-4501.
(3) Shimomura, M.; Kunitake, T., Fluorescence and photoisomerization of azobenzene-containing bilayer membranes. J. Am. Chem. Soc. 1987, 109 (17), 5175-5183.
(4) Kuiper, J. M.; Engberts, J. B., H-aggregation of azobenzene-substituted amphiphiles in vesicular membranes. Langmuir 2004, 20 (4), 1152-1160.
(5) Zhu, Y.; Fujiwara, M., Installing dynamic molecular photomechanics in mesopores: a multifunctional controlled-release nanosystem. Angew. Chem. Int. Ed. 2007, 46 (13), 2241-2244.
(6) Zhang, S.; Wang, C.; Chang, H.; Zhang, Q.; Cheng, Y., Off-on switching of enzyme activity by near-infrared light-induced photothermal phase transition of nanohybrids. Sci. Adv. 2019, 5 (8), eaaw4252.
(7) Toettcher, J. E.; Weiner, O. D.; Lim, W. A., Using optogenetics to interrogate the dynamic control of signal transmission by the Ras/Erk module. Cell 2013, 155 (6), 1422-1434.
(8) Zhou, X. X.; Fan, L. Z.; Li, P.; Shen, K.; Lin, M. Z., Optical control of cell signaling by single-chain photoswitchable kinases. Science 2017, 355 (6327), 836-842.
(9) Wang, L.; Li, Q., Photochromism into nanosystems: towards lighting up the future nanoworld. Chem. Soc. Rev. 2018, 47 (3), 1044-1097.
(10) Timko, B. P.; Arruebo, M.; Shankarappa, S. A.; McAlvin, J. B.; Okonkwo, O. S.; Mizrahi, B.; Stefanescu, C. F.; Gomez, L.; Zhu, J.; Zhu, A., Near-infrared-actuated devices for remotely controlled drug delivery. Proc. Natl. Acad. Sci. USA 2014, 111 (4), 1349-1354.
(11) Chen, Z.; Zhao, Y.; Liu, Y., Advanced Strategies in Enzyme Activity Regulation for Biomedical Applications. ChemBioChem 2022, e202200358.
(12) Aggarwal, K.; Kuka, T. P.; Banik, M.; Medellin, B. P.; Ngo, C. Q.; Xie, D.; Fernandes, Y.; Dangerfield, T. L.; Ye, E.; Bouley, B., Visible light mediated bidirectional control over carbonic anhydrase activity in cells and in vivo using azobenzenesulfonamides. J. Am. Chem. Soc. 2020, 142 (34), 14522-14531.
(13) Mogaki, R.; Okuro, K.; Aida, T., Adhesive photoswitch: selective photochemical modulation of enzymes under physiological conditions. J. Am. Chem. Soc. 2017, 139 (29), 10072-10078.
(14) Chai, J.; Zhao, Y.; Xu, L.; Li, Q.; Hu, X. Y.; Guo, D. S.; Liu, Y., A Noncovalent Photoswitch for Photochemical Regulation of Enzymatic Activity. Angew. Chem. 2022, e202116073.
(15) Schierling, B.; Noël, A.-J.; Wende, W.; Hien, L. T.; Volkov, E.; Kubareva, E.; Oretskaya, T.; Kokkinidis, M.; Römpp, A.; Spengler, B., Controlling the enzymatic activity of a restriction enzyme by light. Proc. Natl. Acad. Sci. USA 2010, 107 (4), 1361-1366.
(16) Cummings, C.; Murata, H.; Koepsel, R.; Russell, A. J., Tailoring enzyme activity and stability using polymer-based protein engineering. Biomaterials 2013, 34 (30), 7437-7443.
(17) Chen, A. H.; Silver, P. A., Designing biological compartmentalization. Trends Cell Biol. 2012, 22 (12), 662-670.
(18) Zhao, Y.; Li, Q.; Chai, J.; Liu, Y., Cargo-Templated Crosslinked Polymer Nanocapsules and Their Biomedical Applications. Adv. NanoBiomed Res. 2021, 1 (4), 2000078.
(19) Dey, S.; Dorey, A.; Abraham, L.; Xing, Y.; Zhang, I.; Zhang, F.; Howorka, S.; Yan, H., A reversibly gated protein-transporting membrane channel made of DNA. Nat. Commun. 2022, 13 (1), 1-12.
(20) Graff, A.; Winterhalter, M.; Meier, W., Nanoreactors from polymer-stabilized liposomes. Langmuir 2001, 17 (3), 919-923.
(21) Van Gelder, P.; Dumas, F.; Winterhalter, M., Understanding the function of bacterial outer membrane channels by reconstitution into black lipid membranes. Biophys. Chem. 2000, 85 (2-3), 153-167.
(22) Schirmer, T., General and specific porins from bacterial outer membranes. J. Struct. Biol. 1998, 121 (2), 101-109.
(23) Li, Y.; Hu, Y.; Wang, J.; Liu, X.; Zhang, W.; Sun, L., Structural insights into a plant mechanosensitive ion channel MSL1. Cell Rep. 2020, 30 (13), 4518-4527. e3.
(24) Atkinson, L.; Milligan, C.; Buckley, N. J.; Deuchars, J., An ATP-gated ion channel at the cell nucleus. Nature 2002, 420 (6911), 42-42.
(25) Gadsby, D. C., Ion channels versus ion pumps: the principal difference, in principle. Nat. Rev. Mol. Cell Biol. 2009, 10 (5), 344-352.
(26) Gouaux, E.; MacKinnon, R., Principles of selective ion transport in channels and pumps. Science 2005, 310 (5753), 1461-1465.
(27) Yang, X.; Chang, J.; Jiang, Y.; Xu, Q.; Wang, M.; Mao, L., In Vivo Activation of Pro-Protein Therapeutics via Chemically Engineered Enzyme Cascade Reaction. CCS Chem. 2021, 3 (2), 780-790.
(28) Xie, G.; Li, P.; Zhao, Z.; Kong, X. Y.; Zhang, Z.; Xiao, K.; Wang, H.; Wen, L.; Jiang, L., Bacteriorhodopsin-inspired light-driven artificial molecule motors for transmembrane mass transportation. Angew. Chem. Int. Ed. 2018, 57 (51), 16708-16712.
(29) Bandara, H. D.; Burdette, S. C., Photoisomerization in different classes of azobenzene. Chem. Soc. Rev. 2012, 41 (5), 1809-1825.
(30) Merino, E.; Ribagorda, M., Control over molecular motion using the cis-trans photoisomerization of the azo group. Beilstein J. Org. Chem. 2012, 8 (1), 1071-1090.
(31) Chen, Y.; Ke, G.; Ma, Y.; Zhu, Z.; Liu, M.; Liu, Y.; Yan, H.; Yang, C. J., A synthetic light-driven substrate channeling system for precise regulation of enzyme cascade activity based on DNA origami. J. Am. Chem. Soc. 2018, 140 (28), 8990-8996.
(32) Krekiehn, N.; Miller, M.; Jung, U.; Ulrich, S.; Herges, R.; Magnussen, O., UV/Vis spectroscopy studies of the photoisomerization kinetics in self-assembled azobenzene-containing adlayers. Langmuir 2015, 31 (30), 8362-8370.
(33) Molla, M. R.; Rangadurai, P.; Antony, L.; Swaminathan, S.; de Pablo, J. J.; Thayumanavan, S., Dynamic actuation of glassy polymersomes through isomerization of a single azobenzene unit at the block copolymer interface. Nat. Chem. 2018, 10 (6), 659-666.
(34) Tecilla, P.; Bonifazi, D., Configurational selection in azobenzene-based supramolecular systems through dual-stimuli processes. ChemistryOpen 2020, 9 (5), 538-553.
(35) Zhao, Y.; Jiang, Y.; Chai, J.; Huang, F.; Zhang, Z.; Liu, Q.; Yang, Z.; Liu, Y.; Shi, L., Neuroprotective nanoscavenger induces coaggregation of β-amyloid and facilitates its clearance in Alzheimer's disease brain. CCS Chem. 2021, 3 (8), 2316-2330.
(36) Abuin, E.; Lissi, E.; Ahumada, M., Diffusion of hydrogen peroxide across DPPC large unilamellar liposomes. Chem. Phys. Lipids 2012, 165 (6), 656-661.
(37) Zhao, Y.; Hou, X.; Chai, J.; Zhang, Z.; Xue, X.; Huang, F.; Liu, J.; Shi, L.; Liu, Y., Stapled Liposomes Enhance Cross-Priming of Radio-Immunotherapy. Adv. Mater. 2022, 34 (3), 2107161.
(38) Kim, B.; Sun, S.; Varner, J. A.; Howell, S. B.; Ruoslahti, E.; Sailor, M. J., Securing the payload, finding the cell, and avoiding the endosome: peptide-targeted, fusogenic porous silicon nanoparticles for delivery of siRNA. Adv. Mater. 2019, 31 (35), 1902952.
(39) Zhao, Y.; Chen, Z.; Li, Q.; Cao, X.; Huang, Q.; Shi, L.; Liu, Y., Polymer-Reinforced Liposomes Amplify Immunogenic Cell Death-Associated Antitumor Immunity for Photodynamic-Immunotherapy. Adv. Funct. Mater. 2022, 2209711.
(40) Chander, N.; Morstein, J.; Bolten, J. S.; Shemet, A.; Cullis, P. R.; Trauner, D.; Witzigmann, D., Optimized photoactivatable lipid nanoparticles enable red light triggered drug release. Small 2021, 17 (21), 2008198.
(41) Suchyta, D. J.; Schoenfisch, M. H., Controlled release of nitric oxide from liposomes. ACS Biomater. Sci. Eng. 2017, 3 (9), 2136-2143.
(42) Fan, W.; Lu, N.; Huang, P.; Liu, Y.; Yang, Z.; Wang, S.; Yu, G.; Liu, Y.; Hu, J.; He, Q., Glucose-responsive sequential generation of hydrogen peroxide and nitric oxide for synergistic cancer starving-like/gas therapy. Angew. Chem. 2017, 129 (5), 1249-1253.
(43) Wang, Y. X. J.; Zhu, X. M.; Liang, Q.; Cheng, C. H.; Wang, W.; Leung, K. C. F., In vivo chemoembolization and magnetic resonance imaging of liver tumors by using iron oxide nanoshell/doxorubicin/poly (vinyl alcohol) hybrid composites. Angew. Chem. 2014, 126 (19), 4912-4915.
(44) Wang, C.; Ye, Y.; Hochu, G. M.; Sadeghifar, H.; Gu, Z., Enhanced cancer immunotherapy by microneedle patch-assisted delivery of anti-PD1 antibody. Nano Lett. 2016, 16 (4), 2334-2340.
(45) Geng, J.; Li, W.; Zhang, Y.; Thottappillil, N.; Clavadetscher, J.; Lilienkampf, A.; Bradley, M., Radical polymerization inside living cells. Nat. Chem. 2019, 11 (6), 578-586.
(46) Zhou, L.; Fan, Y.; Liu, Z.; Chen, L.; Spruijt, E.; Wang, Y., A multiresponsive transformation between surfactant-based coacervates and vesicles. CCS Chem. 2021, 3 (12), 358-366.
a1. Gilleron, J. et al. Image-based analysis of lipid nanoparticle-mediated siRNA delivery, intracellular trafficking and endosomal escape. Nat. Biotechnol. 31, 638-646 (2013).
a2. Kwon, Y. J. Before and after endosomal escape: roles of stimuli-converting siRNA/polymer interactions in determining gene silencing efficiency. Acc. Chem. Res. 45, 1077-1088 (2012).
a3. Zhao, Y. et al. Lipidoid Artificial Compartments for Bidirectional Regulation of Enzyme Activity through Nanomechanical Action. J. Am. Chem. Soc. (2022).
a4. Feng, Y. et al. Molecular pumps and motors. J. Am. Chem. Soc. 143, 5569-5591 (2021).
a5. Rifaie-Graham, O. et al. Photoswitchable gating of non-equilibrium enzymatic feedback in chemically communicating polymersome nanoreactors. Nat. Chem. 15, 110-118 (2023).
a6. Balzani, V., Credi, A., Raymo, F. M. & Stoddart, J. F. Artificial molecular machines. Angew. Chem. Int. Ed. 39, 3348-3391 (2000).
a7. Watson, M. A. & Cockroft, S. L. Man-made molecular machines: membrane bound. Chem. Soc. Rev. 45, 6118-6129 (2016).
a8. Krause, S. & Feringa, B. L. Towards artificial molecular factories from framework-embedded molecular machines. Nat. Rev. Chem. 4, 550-562 (2020).
a9. Gadsby, D. C. Ion channels versus ion pumps: the principal difference, in principle. Nat. Rev. Mol. Cell Biol. 10, 344-352 (2009).
a10. Saper, G. & Hess, H. Synthetic systems powered by biological molecular motors. Chem. Rev. 120, 288-309 (2019).
a11. Chari, A. & Fischer, U. Cellular strategies for the assembly of molecular machines. Trends Biochem. Sci 35, 676-683 (2010).
a12. Pooler, D. R., Lubbe, A. S., Crespi, S. & Feringa, B. L. Designing light-driven rotary molecular motors. Chem. Sci. 12, 14964-14986 (2021).
a13. Simon, J., Schwalm, M., Morstein, J., Trauner, D. & Jasanoff, A. Mapping light distribution in tissue by using MRI-detectable photosensitive liposomes. Nat. Biomed. Eng. 7, 313-322 (2023).
a14. Xie, G. et al. Bacteriorhodopsin-inspired light-driven artificial molecule motors for transmembrane mass transportation. Angew. Chem. Int. Ed. 57, 16708-16712 (2018).
a15. ur Rehman, Z., Zuhorn, I. S. & Hoekstra, D. How cationic lipids transfer nucleic acids into cells and across cellular membranes: recent advances. J. Control Release 166, 46-56 (2013).
a16. Cheng, X. & Lee, R. J. The role of helper lipids in lipid nanoparticles (LNPs) designed for oligonucleotide delivery. Adv. Drug Delivery Rev. 99, 129-137 (2016).
a17. Lechanteur, A. et al. Cationic liposomes carrying siRNA: impact of lipid composition on physicochemical properties, cytotoxicity and endosomal escape. Nanomaterials 8, 270 (2018).
a18. Huang, X. et al. The landscape of mRNA nanomedicine. Nat. Med. 28, 2273-2287 (2022).
a19. Sahin, U., Karikó, K. & Türeci, Ö. mRNA-based therapeutics-developing a new class of drugs. Nat. Rev. Drug Discovery 13, 759-780 (2014).
a20. Doria-Rose, N. et al. Antibody persistence through 6 months after the second dose of mRNA-1273 vaccine for Covid-19. N. Engl. J. Med. 384, 2259-2261 (2021).
a21. Barrière, J. et al. Impaired immunogenicity of BNT162b2 anti-SARS-CoV-2 vaccine in patients treated for solid tumors. Ann. Oncol. 32, 1053-1055 (2021).
a22. Manisty, C. et al. Antibody response to first BNT162b2 dose in previously SARS-CoV-2-infected individuals. The Lancet 397, 1057-1058 (2021).
a23. Tang, Z. et al. A materials-science perspective on tackling COVID-19. Nat. Rev. Mater. 5, 847-860 (2020).
a24. Huang, X. et al. Nanotechnology-based strategies against SARS-CoV-2 variants. Nat. Nanotechnol. 17, 1027-1037 (2022).
a25. Geng, J. et al. Radical polymerization inside living cells. Nat. Chem. 11, 578-586 (2019).
a26. Agasti, S. S. et al. Photoregulated release of caged anticancer drugs from gold nanoparticles. J. Am. Chem. Soc. 131, 5728-5729 (2009).
a27. Brendel, J. C. et al. Secondary Self-Assembly of Supramolecular Nanotubes into Tubisomes and Their Activity on Cells. Angew. Chem. Int. Ed. 57, 16678-16682 (2018).
a28. Zhou, K. et al. Modular degradable dendrimers enable small RNAs to extend survival in an aggressive liver cancer model. Proc. Natl. Acad. Sci. USA 113, 520-525 (2016).
a29. Li, S. et al. Payload distribution and capacity of mRNA lipid nanoparticles. Nat. Commun. 13, 5561 (2022).
a30. Vest, R. T. et al. Small molecule C381 targets the lysosome to reduce inflammation and ameliorate disease in models of neurodegeneration. Proc. Natl. Acad. Sci. USA 119, e2121609119(2022).
a31. Pai, C.-L., Chen, Y.-C., Hsu, C.-Y., Su, H.-L. & Lai, P.-S. Carbon nanotube-mediated photothermal disruption of endosomes/lysosomes reverses doxorubicin resistance in MCF-7/ADR cells. J. Biomed. Nanotechnol. 12, 619-629 (2016).
a32. Thacker, S. G. et al. High-density lipoprotein reduces inflammation from cholesterol crystals by inhibiting inflammasome activation. Immunology 149, 306-319 (2016).
a33. Li, Y., Hu, D., Qi, J., Cui, S. & Chen, W. Lysosomal Reacidification Ameliorates Vinyl Carbamate-Induced Toxicity and Disruption on Lysosomal pH. J. Agric. Food. Chem. 68, 8951-8961 (2020).
a34. Zhang, Y., Li, X., Grassmé, H., Döring, G. & Gulbins, E. Alterations in ceramide concentration and pH determine the release of reactive oxygen species by Cftr-deficient macrophages on infection. J. Immunol. 184, 5104-5111 (2010).
a35. Li, X. et al. Inducing Autophagy and Blocking Autophagic Flux via a Virus-Mimicking Nanodrug for Cancer Therapy. Nano Lett. 22, 9163-9173 (2022).
a36. Pols, M. S. et al. hVps41 and VAMP7 function in direct TGN to late endosome transport of lysosomal membrane proteins. Nat. Commun. 4, 1361 (2013).
a37. Endo, Y., Furuta, A. & Nishino, I. Danon disease: a phenotypic expression of LAMP-2 deficiency. Acta Neuropathol. 129, 391-398 (2015).
a38. Teo, S. L. et al. Unravelling cytosolic delivery of cell penetrating peptides with a quantitative endosomal escape assay. Nat. Commun. 12, 3721 (2021).
a39. Chen, G. et al. A biodegradable nanocapsule delivers a Cas9 ribonucleoprotein complex for in vivo genome editing. Nat. Nanotechnol. 14, 974-980 (2019).
a40. Wang, M. et al. Efficient delivery of genome-editing proteins using bioreducible lipid nanoparticles. Proc. Natl. Acad. Sci. USA 113, 2868-2873 (2016).
a41. Qiu, M., Li, Y., Bloomer, H. & Xu, Q. Developing biodegradable lipid nanoparticles for intracellular mRNA delivery and genome editing. Acc. Chem. Res. 54, 4001-4011 (2021).
a42. Li, Y. et al. Protein and mRNA Delivery Enabled by Cholesteryl-Based Biodegradable Lipidoid Nanoparticles. Angew. Chem. 132, 15067-15074 (2020).
a43. Zuris, J. A. et al. Cationic lipid-mediated delivery of proteins enables efficient protein-based genome editing in vitro and in vivo. Nat. Biotechnol. 33, 73-80 (2015).
a44. Wculek, S. K. et al. Dendritic cells in cancer immunology and immunotherapy. Nat. Rev. Immunol. 20, 7-24 (2020).
a45. Yang, P. et al. Engineering dendritic-cell-based vaccines and PD-1 blockade in self-assembled peptide nanofibrous hydrogel to amplify antitumor T-cell immunity. Nano Lett. 18, 4377-4385 (2018).
a46. Gong, N. et al. Proton-driven transformable nanovaccine for cancer immunotherapy. Nat. Nanotechnol. 15, 1053-1064 (2020).
a47. García-López, V. et al. Molecular machines open cell membranes. Nature 548, 567-572 (2017).

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### OTHER EMBODIMENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of formula (I) or formula (II): 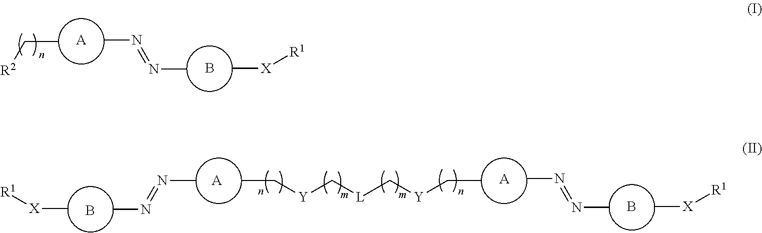
wherein,
A and B are each independently selected from the group consisting of aryl and heteroaryl;
X is independently selected from the group consisting of *—(C═O)NH—, *—NH(C═O)—, —O(C═O)—, and *—(C═O)O—;
Y is independently selected from the group consisting of *—O(C═O)—, *—(C═O)O—, —N(R³)—, and —N⁺(R³)₂—;
L is absent or —NR⁴—;
R¹ is C₆₋₂₀ alkyl;
R² is independently selected from the group consisting of —OR⁵, —O(C═O)R⁵, —(C═O)OR⁵, —N(R³)₂ and —N⁺(R³)₃;
each R³ is independently selected from the group consisting of H and C₁₋₆ alkyl;
R⁴ is independently selected from the group consisting of C₁₋₆ alkyl and C₁₋₆ alkylamino;
R⁵ is independently selected from the group consisting of C₁₋₆ alkyl and C₁₋₆ alkenyl;
n is independently 0-6;
each m is independently 1-6;
* indicates the point of attachment to B; and
** indicates the point of attachment to (CH₂)ₘ.

2. The compound of claim 1, wherein A and B are each phenyl.

3. The compound of claim 1, wherein X is *—NH(C═O)—.

4. The compound of claim 1, wherein R¹ is C₈₋₁₂ alkyl.

5. The compound of claim 4, wherein R¹ is C₁₁ alkyl.

6. The compound of claim 1, wherein Y is —N⁺(R³)₂—.

7. The compound of claim 1, wherein L is absent.

8. The compound of claim 1, wherein n is 1.

9. The compound of claim 1, wherein m is 1.

10. The compound of claim 1, wherein R² is —N⁺(R³)₃.

11. The compound of claim 1, wherein each R³ is methyl.

12. The compound of claim 1, wherein the compound is selected from the group consisting of:

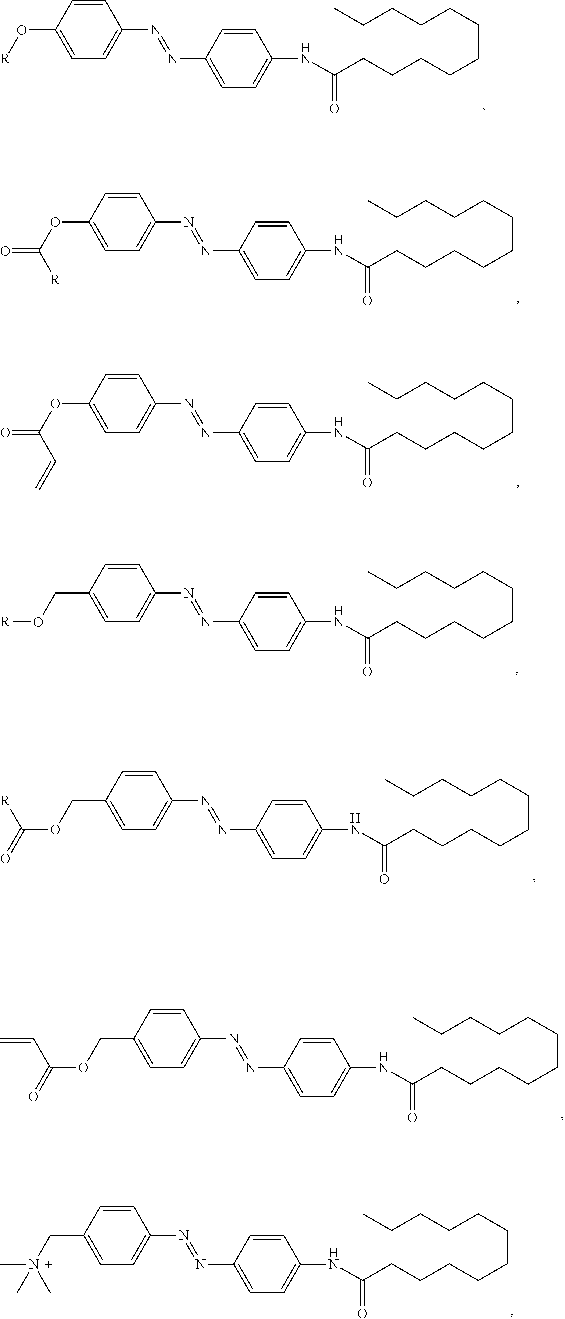

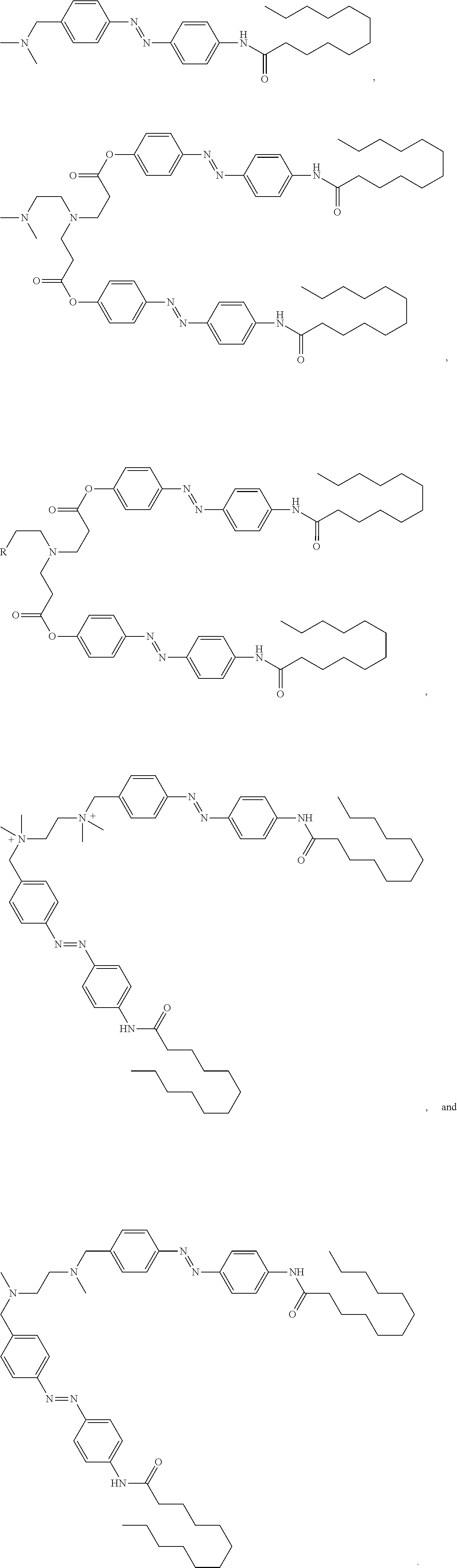

13. The compound of claim 1, wherein the compound is:

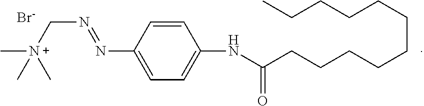 [SMILES: CCCCCCCCCCCC(=O)Nc1ccc(/N=N/C[N+](C)(C)C)cc1 ; [Br-]]

14. The compound of claim 1, wherein the compound is:

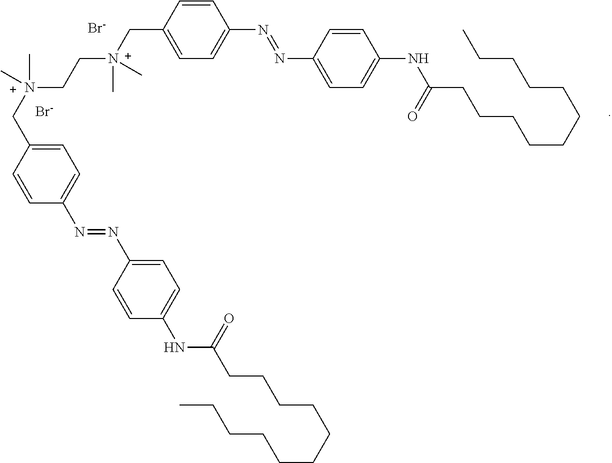 [SMILES: CCCCCCCCCCCC(=O)Nc1ccc(/N=N/c2ccc(C[N+](C)(C)CC[N+](C)(C)Cc3ccc(/N=N/c4ccc(NC(=O)CCCCCCCCCCC)cc4)cc3)cc2)cc1 ; [Br-] ; [Br-]]

15. A lipidoid artificial compartment, comprising a compound of claim 1.

16. The lipidoid artificial compartment of claim 15, wherein the amount of compound of formula (I) or (II) in the lipidoid artificial compartment is about 1 to about 20 mol %.

17. The lipidoid artificial compartment of claim 15, further comprising a helper lipid.

18-20. (canceled)

21. The lipidoid artificial compartment of claim 15, further comprising a surfactant.

22-24. (canceled)

25. The lipidoid artificial compartment of claim 15, further comprising a PEG-modified lipid.

26. (canceled)

27. (canceled)

28. The lipidoid artificial compartment of claim 15, further comprising cholesterol.

29. (canceled)

30. (canceled)
